(19) United States
(12) Patent Application Publication
(10) Pub. No.: US 2024/0052099 A1
(43) Pub. Date: Feb. 15, 2024

(54) POLYANHYDRIDES AND METHODS OF MAKING SAME

(71) Applicant: POLYGENE LTD., Jerusalem (IL)

(72) Inventor: Abraham Jakob DOMB, Jerusalem (IL)

(21) Appl. No.: 18/040,434

(22) PCT Filed: Aug. 5, 2021
(86) PCT No.: PCT/IL2021/050953

§ 371 Date: Feb. 3, 2023

Related U.S. Application Data
(60) Provisional application No. 63/062,563, filed on Aug. 7, 2020.

(51) Int. Cl. C08G 67/04; A61K 31/343; A61K 47/34

(52) U.S. Cl. CPC C08G 67/04; A61K 31/343; A61K 47/34

(57) ABSTRACT

The invention disclosed herein concerns a process for producing a polyanhydride of a narrow-polydispersity and uses of the polyanhydride.

### FIELD OF THE INVENTION

The present invention generally relates to polyanhydrides with tuneable properties and to methods for their preparation.

### BACKGROUND OF THE INVENTION

Polyanhydrides have been investigated as carriers for the controlled delivery of several drugs [1]. Polyanhydrides are desirable as controlled release careers because of their surface eroding properties.

Even though polyanhydrides are easy and inexpensive to synthesize and scale up, they exhibit a short shelf-life. They have inherent high reactivity toward water, which prompts rapid hydrolytic degradation. Due to the high rate of hydrolysis, polyanhydrides endure surface erosion rather than bulk degradation. They are also prone to depolymerisation via anhydride interchange during storage. Hence, polyanhydrides need to be kept at freezing storage conditions that restricting their usage in drug delivery products.

To overcome some of the known deficiencies, alternating polyanhydride copolymers have been utilized. The class of poly(ester-anhydride)s exhibits better drug release profile, however, the shelf life of the polymer does not substantially improve. Polyanhydrides based on ε-caprolactone were found to suffer from enhanced hydrolytic stability with a limited shelf life.

Polyanhydrides are commonly prepared by poly-condensation of dicarboxylic acids in a large excess of refluxing acetic anhydride. For example, Jaszcz at al., [2] synthesized a polyanhydride using 1:10 w/v acetic anhydride; Narasimhan et al., [3] synthesized several polyanhydrides using 1:45 w/v acetic anhydride; I Ming Chu [4] polymerized sebacic acid and other diacids using 1:10 w/v acetic anhydride; and A P Herrera et al., [5] synthesized poly(azelaic ahydride) by microwave irradiation (5 minutes) using a 1:3 w/v relation of solid dicarboxilic acid to acetic anhydride. Early reports on polyahydride synthesis used 1:10 w/v diacid to anhydride ratio, in some cases a 1:5 ratio is reported. Thus, it is noted that in all available methodologies, excess of acetic anhydride of 3 to 10 w/v times have been used which formed polymers of uncontrolled molecular weights and polydispersity.

One such example is the poly(ester-anhydride) based on the ricinoleic acid and sebacic acid reported in [6-7] and [10].

### REFERENCES

[1] Adv. Mater., 30 (2018) 1706815
[2] RSC Adv., 2019, 9, 20892
[3] ACS Biomater. Sci. Eng. 2020, 6, 265-276
[4] J Polym Res 26, 1 (2019)
[5] J. Physics: Conf. Series 687 (2016) 012049
[6] U.S. Pat. No. 10,774,176
[7] US 2020/0101163
[8] U.S. Pat. No. 4,888,176
[9] J. Polymer Sci. Part a-Poly. Chem. 1987; 25(12):3373-3386
[10] Domb et al., 2017, J of Controlled Release, 257, 156-162

### SUMMARY OF THE INVENTION

Polyanhydrides are a class of biodegradable polymers characterized by anhydride bonds that connect repeat units of a polymer backbone chain. Despite the extensive use of polyanhydrides and the various methodologies that have been utilized for their preparation, as indicated in the background of the invention, polyanhydrides remain generally prone to hydrolysis, depolymerization and may therefore be produced along with decomposition products which make their usability in the field such as the medical fields less attractive.

The inventors of the technology disclosed herein have developed a methodology for producing polyanhydrides with improved properties to those previously disclosed in the prior art. The novel methodology of the invention involves a reaction between a hydroxyl-acid with a dicarboxylic acid or cyclic anhydride to produce in one pot a narrow disperse polyanhydride at high reproducibility. Unlike the synthesis leading to the polydisperse polymers of the art, such as [6] or [7] or [10], narrow disperse polymers with high reproducibility of molecular weight were prepared by avoiding use of polymerized precursors. By reacting the polymer units directly in the presence of small amounts of an acetylation agent, and in absence of a solvent, polymerization of the polymer units was achieved with great repeatability and uniformity. The narrow dispersed polymer was manufactured once and again with low or no variability in polymer physical properties, composition molecular weight and purity.

Thus, in a first aspect of the invention there is provided a process for producing a polyanhydride of a narrow-polydispersity.

Also provided is a process for producing a narrow-polydispersed polyanhydride, the process comprising melt polycondensation of a dicarboxylic acid and a hydroxyl-alkanoic acid in the presence of an amount of acetic anhydride not exceeding a mole equivalent thereof per each free carboxylic acid group and in absence of a solvent.

As will be detailed further below, the process of the invention does not utilize any solvent medium to carry out condensation of the material precursors into the polyanhydride. No solvent, aqueous or organic, is used for the conversion. Thus, the materials are thermally transformed in the melt (herein “melt polycondensation”). Within the context of the process disclosed herein, the material precursors used to manufacture the narrow-dispersed polyanhydrides do not act as solvents and thus are not to be considered solvents.

The absence of a solvent and the sequential addition of the various precursors, allows for producing a final product that is well characterized and reproducible to meet regulatory requirements of the highest standards and which exhibits narrow polydispersity. The term “narrow polydispersity” or any lingual variation thereof, when made in reference to a polyanhydride according to the invention, defines a collection of materials having substantially identical compositions (type of repeating groups and manner of repetition) and molecular weights. The narrow polydispersity of polyanhydrides of the invention, defined by the ratio Mw/Mn (wherein Mw is the weight-average molecular weight and Mn is the number-average molecular weight) is below 2.5 or below 2. Putting it differently, the narrow disperse or narrow polydisperse polyanhydrides of the invention have a polydispersity value of no more than 2.5 or 2 (or a value between 2.5 and 1, or between 2 and 1). As a person of skill would appreciate these values indicate a very small variation and thus very narrow dispersity, suggesting a polymer that is nearly monodisperse.

As a person of skill would also appreciate the reported polydispersity values of polyanhydrides range between 3 and 9, values that suggest high dispersity of molecular weights. For example, publications [8] and [9] reporting on polyanhydrides of a molecular weight exceeding 5,000, reported polydispersity values ranging from about 4 to about 9.

Polyanhydrides of the invention also exhibit high reproducibility, namely a reproducibility in polymer molecular weight that is no more than 30% deviation from polymer average molecular weight.

The term “in absence of a solvent” herein refers to the property of the process of the invention as having no or a minute amount of solvent(s) that may be derived from impurities present with the precursor materials. Such impurities will not exceed 0.001%, 0.005%, 0.01%, 0.05% or 0.1% (w/w) of the total weight of the reaction materials used. Processes of the invention do not comprise and exclude steps of using or adding a solvent, or steps which provide raw materials or intermediate materials dissolved in a solvent.

The process of the invention comprises:
reacting a dicarboxylic acid (DA) or a cyclic anhydride with an hydroxy-alkanoic acid (HA) under conditions permitting esterification of the dicarboxylic acid (to obtain a mono ester of the dicarboxylic acid or a di-ester thereof or a mixture thereof); and
transforming the esterified dicarboxylic acid (mono or di- or mixture thereof) into the narrow-polydisperse polyanhydride.

The process of the invention permits for direct condensation in bulk (in the melt), without a pre-reaction to form a polymer or an oligomer of any of the material precursors used. In an exemplary process, sebacic acid (SA) (a dicarboxylic acid) was reacted with ricinoleic acid (RA) (a hydroxyl-alkanoic acid) at a 30:70 w/w ratio to form a mixture of SA-RA dimers and RA-SA-RA trimers with minimal or no RA or RA-RA ester molecules in the reaction product. The SA-RA and RA-SA-RA mixture (free of the precursor molecules and of the RA-RA molecules) is thereafter treated with no more than one molar equivalent of acetic anhydride per free carboxylic acid group (being typically 2 free carboxylic acid groups and thus no more than 2 molar equivalents) to acetylate the free ester and thereafter polymerize the acetylated segments into the narrow-dispersed polyanhydride having the repeating . . . RA-SA-RA-SA . . . sequence. The process is depicted in FIG. 1.

All processes, methods and preparation steps of the herein disclosed invention are free of or exclude use of a poly(dicarboxylic acid), e.g., poly sebacic acid. None of the processes, methods and preparations of the invention comprises a step which comprises or consists formation or use of poly(dicarboxylic acid), e.g., poly sebacic acid. Thus, a process of the invention may be generally considered to comprise a. melt condensation of SA and RA (or equivalents thereof) to form dicarboxylic acid oligomers; b. oligomer activation with acetic anhydride; c. melt polycondensation to form a polyanhydride, wherein the preparation does not comprise use of poly(dicarboxylic acid), e.g., poly sebacic acid. The first step is obtaining each of the dicarboxylic acid, e.g., SA and the hydroxyl-alkanoic acid, e.g., RA and subsequently forming their oligomers (dimer or trimer as defined herein) or the first step may be melt condensation of e.g., SA and RA to form dicarboxylic acid oligomers. Thus a first step in processes, methods, and preparations of the invention is not formation of a poly(dicarboxylic acid), e.g., poly sebacic acid. As used herein, the poly(dicarboxylic acid), e.g., poly sebacic acid is a polymer or an oligomer consisting the polycarboxylic acid or SA (wherein the terminal oxygen groups may be acetylated or free).

Apart from SA and RA other dicarboxylic acids and hydroxy-alkanoic acids may be used. In some embodiments, the dicarboxylic acid (DA) is a dicarboxylic acid of a C₆-C₁₅ alkylene or alkenylene. Non-limiting examples include succinic acid, adipic acid, maleic acid, suberic acid, sebacic acid (SA), decandioic acid, azelaic acid, undecanedioic acid, dodecanedioic acid, brassylic acid, phthalic acid and others. In some embodiments, the dicarboxylic acid is a cyclic anhydride of the aforementioned dicarboxylic acids or diacids such as succinic acid, maleic acid and phthalic acid.

The hydroxy-alkanoic acids (HA) are typically alkanes or alkenes having a carboxylic acid group and a hydroxyl group, wherein, in some embodiments, the carboxylic acid is an end of chain group and the hydroxyl group is positioned along the carbon chain. The hydroxyl groups is, in some embodiments, positioned around the middle of the chain, such that when esterification occurs, presence of an alkyl or alkenyl chain segments in the vicinity of the ester bond protects the bond from hydrolysis. This is well depicted and exemplified in the structure of the final polyanhydride, shown in FIG. 1, and reproduced below, formed between SA and RA. The arrows mark the alkyl chain segments in the vicinity of the ester bonds which protect the ester bond from hydrolysis.

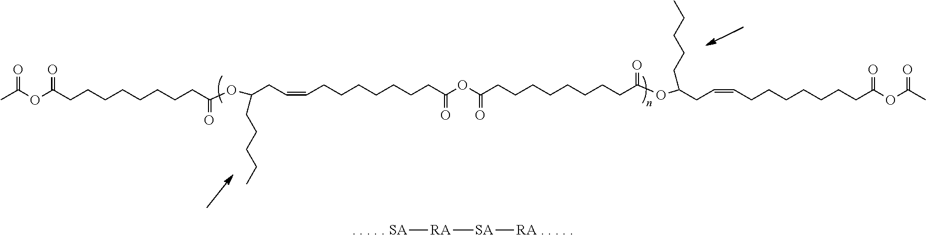

Thus, in a hydroxyl-alkanoic acid (HA) used in accordance with the invention, the hydroxyl group is not a terminal group, namely not positioned on the carbon atom at the end of the chain. The hydroxyl group is typically positioned 1, 2, 3, 4, 5, 6, 7, or 8 carbon atoms away (depending on the length of the alkanoic chain) from the terminal atom (counted from the terminal atom not bearing the carboxylic acid group).

In cases a double bond is present it may be one or more double bonds positioned between any two carbon atoms of the main alkanoic carbon chain. In some embodiments, the double bond may be positioned between the carboxylic acid group and the hydroxyl group. In other embodiments, the double bond is present between the hydroxyl group and the terminus carbon of the alkanoic chain. The number of double bonds may be 1 or more. In some embodiments, depending on the length of the carbon chain, the number of double bonds may be 1, 2, or 3.

In some embodiments, the alkanoic chain comprises between 2 and 15 carbon atoms. Non-limiting examples of such hydroxyl-alkanoic acid is ricinoleic acid (RA), hydroxystearic acid (HSA), hydroxyoctanoic acid, hydroxydecanoic acids, hydroxydodecanoic acid, lactic acid, glycolic acid, hyroxycaproic acid and others.

In some embodiments, the polyanhydride is formed of a dicarboxylic acid such as succinic acid, or adipic acid, or maleic acid, or suberic acid, or sebacic acid (SA), or decandioic acid, or azelaic acid, or undecanedioic acid, or dodecanedioic acid, or brassylic acid, phthalic acid, and a hydroxy-alkanoic acid such as ricinoleic acid (RA), ricinoleic acid (RA), hydroxystearic acid (HSA), hydroxyoctanoic acid, hydroxydecanoic acids, hydroxydodecanoic acid, lactic acid, glycolic acid, or hyroxycaproic acid.

In some embodiments, the polyanhydride is formed of SA. In some embodiments, the polyanhydride is formed of RA. In some embodiments, the polyanhydride is formed of RA and SA, as disclosed herein. Putting it differently, in some embodiments, the dicarboxylic acid is SA, and in other embodiments, the hydroxyl-alkanoic acid is RA. In further embodiments, the dicarboxylic acid is RA and the hydroxyl-alkanoic acid is SA.

The weight ratio of dicarboxylic acid:hydroxyl-alkanoic acid is dependent on the molecular weight of each unit. Mixture of dimers and trimers of dicarboxylic acid and hydroxyl-alkanoic acid can be used to form a heterogeneous polymer consisting anhydride bonds and ester bonds between dicarboxylic acids and hydroxyl-alkanoic acid units with minimal ester bonds between two hydroxyl-alkanoic acids. On the other hand, formation of anhydride diads of the dicarboxylic acid monomers along the polymer chain, may limit the storage stability of the polymer. Thus, in a process of the invention, the molar ratio between a dicarboxylic acid (DA) and a hydroxyl-alkanoic acid (HA) is typically equivalent or in favor of the hydroxyl-alkanoic acid. In other words, the amount of the hydroxyl-alkanoic acid is preferably 1:1 to 1:2 molar equivalent relative to the dicarboxylic acid. In some embodiments, the weight ratio DA:HA is 1:1, 1:1.1, 1:1.2, 1:1.3, 1:1.4, 1:1.5, 1:1.6, 1:1.7, 1:1.8, 1:1.9, 1:2, respectively.

In some embodiments, the molar ratio between the dicarboxylic acid and the hydroxyl-alkanoic acid ranges between 1:1 and 1:2, respectively to avoid ester bond formation between hydroxyl-alkanoic acids, so that the polymer comprises anhydride bonds and ester bonds only between a dicarboxylic acid and a hydroxy-alkanoic acid.

In some embodiments, the weight ratio between the dicarboxylic acid and the hydroxyl-alkanoic acid is 30:70, 35:65 or 25:75 for SA and RA building blocks, respectively.

An excess amount of the hydroxyl-alkanoic acid permits mono- and di-esterification of the dicarboxylic acid (with some amount of a mono esterified dicarboxylic acid), and avoids formation of ester dimers of the hydroxyl-alkanoic acid. The DA-HA and HA-DA-HA mixture (herein a “dimer-trimer mixture”) is obtained by heating a mixture of DA and HA, in the indicated ratios, at a temperature above 80° C. In some embodiments, the temperature is between 80 and 200, between 100 and 190, between 100 and 180, between 100 and 170, between 100 and 160, between 100 and 150, between 100 and 140, between 100 and 130, or between 100 and 120° C.

The condensation of the dicarboxylic acid and the hydroxyl-alkanoic acid involves direct ester condensation to provide the dimer-trimer dicarboxylic acid oligomer mixture. The dimer-trimers oligomers are polymerized into a polyanhydride by activation of the carboxylic acid ends with acetic anhydride. The amount of the acetic anhydride used is not greater than one molar equivalent of acetic anhydride per every free carboxylic acid group in the oligomers. The dimer DA-HA has two free carboxylic acid groups. Similarly, the trimer HA-DA-HA has 2 free carboxylic acid groups. Thus, no more than 2 molar equivalents of acetic anhydride may be used. In some embodiments, the amount of acetic anhydride is 2, 1.9, 1.8, 1.7, 1.6, 1.5, 1.4, 1.3, 1.2 or 1.1 molar equivalents.

In some embodiments, the acetylation step may be carried out at a temperature above 40° C. In some embodiments, the acetylation temperature is between 40° C. and the boiling point of acetic anhydride. In some embodiments, the acetylation temperature is between 40 and 90, between 40 and 100, between 40 and 110, between 80 and the boiling point of the acylation anhydride. The temperature used for the acylation-activation of the oligomers is a function of time, the longer the reaction time, the lower the temperature to be applied. It is possible to react the diacid oligomers with acetic anhydride under pressure to expedite the reaction or perform the reaction under microwave heating. These methods require tuning the reaction conditions so that the oligomers are acetylated and not deteriorated. Moreover, other acetylation methods may apply, including reaction with acetyl chloride with an acid scavenger.

The temperature may be increased following acetylation to condense the acetylated precursors to form the aforementioned dimer/trimer mixture.

The transforming into the narrow-polydispersed polyanhydride is achieved by polymerization. Polymerization of the dimer-trimer mixture into a polymer of the invention may be achieved by heating the acetylated dimers and trimers under low pressure and elevated temperatures. In some embodiments, polymerization is achievable in vaccuo under heating. The thermal conditions may involve heating the acetylated dimer-trimer mixture to a temperature between 100 and 200, between 100 and 190, between 100 and 180, between 130 and 170, between 130 and 160, between 130 and 150, or between 130 and 140° C. In some embodiments, the temperature is between 120 and 170 or between 130 and 160° C. The reaction time is an important parameter, as the higher the reaction temperature, the shorter is the reaction time. There is a minimum time required for forming the oligomers and polymers, longer reaction time has no or little effect on the oligomer composition or polymer molecular weight. The reaction time is dependent on the batch size and the reaction conditions, including the mixing method and rate and vacuum profile applied.

While in the above description, SA and RA are specifically exemplified, other dicarboxylic acids and hydroxyl-alkanoic acids may be equivalently used. Thus, the above description is by no way limiting.

In some embodiments, polymerization is achievable at high thermal conditions, as specified, and under vacuum.

In some embodiments, the process comprises:
reacting a dicarboxylic acid (DA) or a cyclic anhydride with an hydroxyl-alkanoic acid (HA) at a temperature between 80 and 200° C. to obtain a mixture of a mono ester (DA-HA) and a diester (HA-DA-HA) of the dicarboxylic acid; and
reacting the mixture with acetic anhydride under conditions permitting polymerization of the mono ester and diester into the polyanhydride.

In some embodiments, the process comprises:
reacting a dicarboxylic acid (DA) or a cyclic anhydride with an hydroxyl-alkanoic acid (HA) at a temperature between 80 and 200° C. to obtain a mixture of a mono ester (DA-HA) and a diester (HA-DA-HA) of the dicarboxylic acid; and
reacting the mixture with acetic anhydride to acetylate the mixture of monoester and diester; and
thermally treating the acetylated mixture under conditions permitting polymerization into the polyanhydride.

In some embodiments, the process comprises:
reacting a dicarboxylic acid (DA) or a cyclic anhydride with an hydroxyl-alkanoic acid (HA) in the presence of acetic anhydride at a temperature between 80 and 200° C. to obtain a mixture of a mono ester (DA-HA) and a diester (HA-DA-HA) of the dicarboxylic acid; and
thermally treating the acetylated mixture in vaccuo at a temperature between 100 and 200° C., permitting polymerization to afford the polyanhydride.

The dimer DA-HA is of the structure:

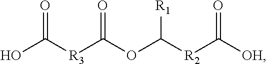

wherein R₁ and R₂ designate the carbon chain of the hydroxy-alkanoic acid and wherein R₃ group designates the carbon chain of the diacid.

The trimer HA-DA-HA is similarly of the structure:

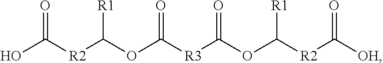

wherein the dicarboxylic acid (R3 being its central carbon chain) is substituted on both ends with a hydroxy-alkanoic acid group.

The polymer of the invention is thus a polyanhydride where the mixture or dimer and trimer dicarboxylic acids are linked to a chain by an anhydride bond. The preferred polymer structure is a polymer chain with no or minimal HA-HA ester bonds or DA-DA-DA anhydride bonds. Processes of the invention exclude such processes which produce polydisperse polyanhydrides. Processes of the invention are free of steps forming or utilizing a polymer or oligomer derived from (consisting) DA or derived from (consisting) HA. One such process is a process utilizing SA and RA and disclosed in publications [6] and [7], as explained hereinabove.

Polyanhydrides of the invention are typically linear compounds. However, processes of the invention may be modified to produce crosslinked polyanhydrides, or branched polyanhydrides. Branching and crosslinking of the polymers may be achieved by adding a polycarboxylic acid such as a tricarboxylic acid into the dimer-trimer mixture, prior to the anhydride acetylation with acetic anhydride. Thus, a process for crosslinking or branching a polyanhydride according to the invention may comprise:
reacting a dicarboxylic acid (DA) or a cyclic anhydride with an hydroxyl-alkanoic acid (HA), under any of the aforementioned stated conditions to obtain a mixture of a mono ester (DA-HA) and a diester (HA-DA-HA) of the dicarboxylic acid;
treating the mixture with at least one polycarboxylic acid; and
reacting the resulting mixture with acetic anhydride under conditions permitting polymerization of the branched or crosslinked esters into the polyanhydride.

Examples of polycaboxylic acid molecules that may be used include citric acid, aconitic acid, isocitric acid, propane-1,2,3-tricarboxylic acid, agaric acid, benzene-tricarboxylic acid, polyacrylic acid and 1,2,3,4,5,6-cyclohexanehexacarboxylic acid.

The amount of the polycarboxylic acid used may range from 0.1 to about 5 wt % to form a branched polymer with higher viscosity compared to the linear polymer or form a crosslinked polymer when using 5% or more polyacid molecules. The physical and mechanical properties of the polymers are affected by the degree of branching or crosslinking.

In another aspect, there is provided a polyanhydride prepared by melt condensation of a dicarboxylic acid and a hydroxyl-alkanoic acid with a mole equivalent or less of acetic anhydride per carboxylic acid.

The invention also contemplates a polyanhydride of the form -(DA-HA)n-, wherein DA is the dicarboxylic acid, HA is the hydroxyl-alkanoic acid and n is an integer designating the number of units DA-HA repeated in the polymer. The polyanhydride of the invention is an alternating DA, HA units. In other words, along the polymer chain, alternating anhydride and ester bonds conjugate between DA and HA moieties with minimal to no ester bond between two neighboring HA units or DA-DA-DA anhydride diads.

In some embodiments, the polyanhydride of a form -(DA-HA)n- is produced by a process according to the invention. Thus, there is provided a polymer of the form -(DA-HA)n-, as defined herein, manufactured by a process comprising:
reacting a dicarboxylic acid or a cyclic anhydride with an hydroxy-alkanoic acid under conditions permitting esterification of the dicarboxylic acid (to obtain a mono ester of the dicarboxylic acid or a di-ester thereof); and
transforming the dicarboxylic acid (mono or di-) esterified into a narrow-dispersed polyanhydride.

All embodiments of the process recited herein are applicable to manufacturing a polymer of the form -(DA-HA)n-, utilizing the aforementioned process.

In some embodiments, DA is SA and HA is RA.

In some embodiments, the number n of repeating DA-HA units is between 10 and 100. The number of repeating units in a polymer of the invention and thus the polymer molecular weight may be tailored by selecting the amount of acetic acid used for the esterification of the polymer precursors, as detailed herein. In some embodiments, the polymers of the invention are of a molecular weight that is between 1,000 and 25,000 Da or between 5,000 and 15,000 Da.

As a person of skill in the art would appreciate, processes of the invention may be carried out in a single pot with only three components, the DA and HA starting materials and a minimal amount of acetic anhydride. Unlike processes of the art, processes of the invention comprise a step of forming the dimer-trimer mixture disclosed above and a step of polymerization. The simplicity of the process and the fact that a narrow-dispersed product is obtained permits full control of polymer molecular weight and thus full control of properties that are affected by the molecular weight. Such properties may be polymer viscosity, injectability, freezing point, degradation rate and dispersibility in water. The ability to control properties of the polymer permits process uniformity and repeatability. Since the polymer composition can be tailored and defined to meet certain regulatory prerequisites, narrow-dispersed polyanhydrides of the invention may be used for delivery of drugs, e.g., by injection. From a production viewpoint, the entire process is continuous with no separation or isolation steps and with only one by-product, acetic anhydride at a minimal amount that saves the cost of starting materials and waste management.

Also provided is a polyanhydride of the form -(SA-RA)n-, wherein SA is sebacic acid and RA is ricinoleic acid, and wherein n is an integer between 10 and 100, prepared by melt condensation of SA and RA with a mole equivalent or less of acetic anhydride per carboxylic acid group, the polyanhydride being a narrow-polydispersed polymer.

Further provided is a process for producing a narrow-polydisperse polyanhydride of the form -(SA-RA)n-, wherein SA is sebacic acid and RA is ricinoleic acid, and wherein n is an integer between 10 and 100, the process comprising melt polycondensation of RA and SA in presence of an amount of acetic anhydride not exceeding a mole equivalent thereof per each free carboxylic acid group and in absence of a solvent.

Also contemplated are the following polyanhydrides and processes for their production:
1) Poly(hydroxyoctanoic acid succinate) (PHOAS) formed of hydroxyoctanoic acid and succinic acid;
2) Poly(hydroxydodecanoic acid succinate) (PHDDAS) formed of hydroxydodecanoic acid and succinic acid;
3) Poly(hydroxystearic acid phthalate) (PHSAP) formed of hydroxystearic acid and phthalic acid;
4) Poly(hydroxystearic acid maleate) (PHSAM) formed of hydroxystearic acid and maleic acid;
5) Poly(hydroxystearic acid succinate) (PHSAS) formed of hydroxystearic acid and succinic acid;
6) Poly(ricinoleic acid phthalate) (PRAP) formed of ricinoleic acid and phthalic acid;
7) Poly(ricinoleic acid maleate) (PRAM) formed of ricinoleic acid and maleic acid;
8) Poly(ricinoleic acid succinate) (PRAS) formed of ricinoleic acid and succinic acid;
9) Poly(sebacic acid-hydroxystearic acid) (P(SA-HSA)) formed of hydroxystearic acid and sebacic acid.

The polymers of the invention may also serve as carriers of at least one agent e.g. an active agent, a drug, a medicament, an additive, a preservative, a coloring agent, a cosmetic agent, an herbicide, an insecticide or a fertilizer, e.g., for improving and protecting plant growth, and others, intended to be released from the carrier in a controlled fashion. Thus, the invention also provides a composition comprising at least one agent, as defined, and a carrier in a form of a polyanhydride of the invention. In some embodiments, the composition is a pharmaceutical composition or a cosmetic composition or an agricultural composition.

Compositions of the invention may be formulated in a variety of methods and into a variety of forms. Accordingly, there is a wide variety of suitable formulations of the composition of the present invention. For therapeutic or cosmetic purposes, compositions of the invention may be formulated for oral, aerosol, parenteral, subcutaneous, intravenous, intramuscular, inter-peritoneal, rectal, and vaginal administrations. In some embodiments, the composition is configured for delivery by injection.

The polymer of the invention may also be implanted subcutaneously, intramuscular, in the brain or in any tissue in the human or animal body and degrade and eliminate from the injection site within months. Thus, an implantable device is provided which comprises a polymer of the invention.

Active agents used in a variety of medical methodologies may be used in compositions and devices of the invention. Such active agents may be present in an amount between 0.1 and 75% w/w, or more, depending on the potency of the drug, its physical and chemical properties, the volume of injection or application and the desired release profile. The hydrophobic nature of the polymer may protect, in part, the incorporated drug from being deteriorated due to light interaction, oxidation or hydrolysis during storage and in the application site (soil, field, patient, etc.). The pasty polymer can be injected into tissue or spread on disease surface such as the lungs and other tissues. The distribution of the active agent into the tissue, after injection, is dependent on the tissue properties; usually the diffusion of the active agent can reach 15 mm from the injection site. The spread of active agent can be improved by adding agents that enhance tissue penetration such as Azone, isopropyl myristate, decyl oleate, oleyl alcohol and triacetin. Other agents that may improve diffusion of active agents within a collagen rich tissue include collagen formation inhibitors such as steroids and losartan. The polymer containing the drug can be dispersed in water to form a dispersion that can be injected or spread into and onto tissue. The release of active agents can be of zero order or first order profile for periods from a few days to about 8 weeks.

### BRIEF DESCRIPTION OF THE DRAWINGS

The invention may be more clearly understood upon reading of the following detailed description of non-limiting exemplary embodiments thereof, with reference to the following drawings, in which:

FIG. 1 is a synthetic scheme of a polyanhydride of the present invention.

FIGS. 2A-B present molecular weight (A) and standard deviation of molecular weight against the amount of acetic anhydride used during the activation step;

FIGS. 3A-B provide a storage stability diagram of polyanhydrides according to the present invention in comparison to other polymers;

FIG. 4 provides a Hydrolytic stability diagram of polyanhydrides in phosphate buffer pH 7.4 at 37° C. according the present invention;

FIG. 5 depicts a variation of the molecular weight of poly(SA-RA) ester-anhydride with 3:7 weight ratio depending upon the acetic anhydride used to activate the monomers according to the present invention;

FIGS. 6A-B shows (FIG. 6A) standard curve of Escitalopram in phosphate buffer pH 7.2; (FIG. 6B) appearance of PSA:RA Escitalopram gel according to the present invention;

FIG. 7 shows a curve of In vitro cumulative release of Escitalopram from PSA:RA 3:7 formulation at 37° C. according to another embodiment of the present invention;

FIG. 8 shows an in vitro release study of PSA-RA duplicate formulation as a function of time.

### DETAILED DESCRIPTION OF THE INVENTION

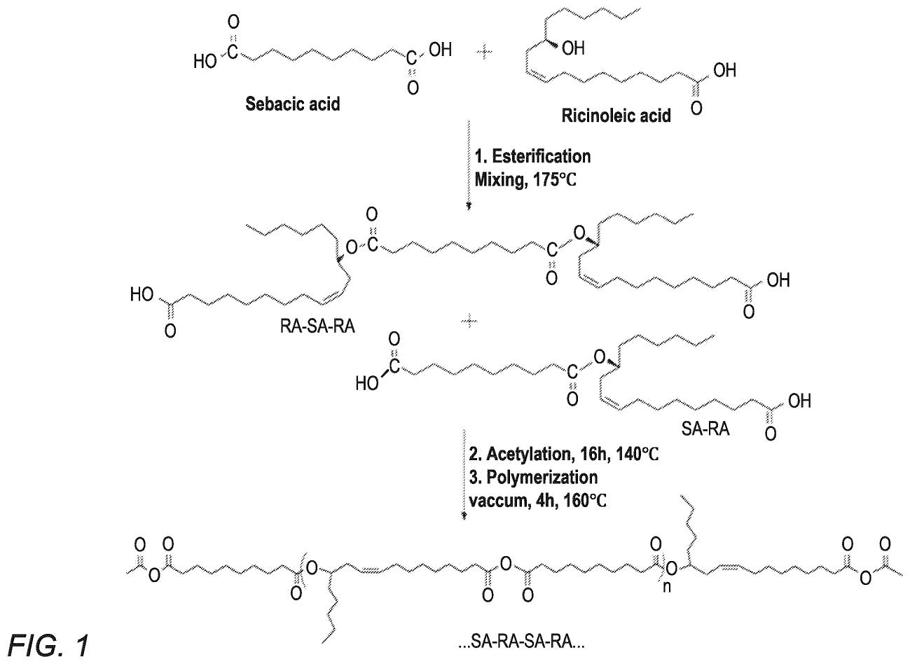

FIG. 1 presents the synthetic pathway for compounds of the invention. Without wishing to be bound by theory, the stability of the polyanhydrides is due to their structure, as presented in FIG. 1, wherein a fatty chain resides near each of the anhydride bonds along the polymer chain that protects the anhydride bonds from being hydrolyzed by residual moisture.

Referring further to FIG. 1, according to some embodiments, after the first step of formation of the RA-SA and RA-SA-RA oligomers by direct condensation, the second and final polymerization step is performed, without the need for any purification. Thus, the second polymerization step may involve adding an equivalent amount (or less) of acetic anhydride to form a solution that is sterile filtered with a filter (e.g. 0.2 micron filter) to remove any bacteria or pyrogens and heated to form the acetylated oligomers where upon application of vacuum and heating (e.g. 160° C.) for several hours.

### Example 1: Controlled Synthesis of Oligomers of Different Type of Dicarboxylic Acid and Hydroxy Acids

Aim: development of an alternative method to synthesis of oligomers of different type of dicarboxylic acid and hydroxy acids.
Materials: Suberic acid (SUA) and dodecanedioic acid (DDDA) were used as received. Ricinoleic acid (RA) was prepared from the hydrolysis of castor oil as described in the synthesis part.

### Spectral Analysis

¹H and ¹³C NMR spectra were obtained on a Varian 300 MHz NMR spectrometer using CDCl₃ as solvent containing tetramethylsilane as shift reference. Fourier transform infrared (FTIR) spectroscopy was performed using a Smart iTR ATR sampling accessory for Nicolet iS10 spectrometer with a diamond crystal (Thermo Scientific, Massachusetts). Preparation of ricinoleic acid from castor oil: In a 1000 mL round bottom flask, 48 g of KOH was dissolved in 400 mL of ethanol by heating (65° C.). Then, 200 g of castor oil was added to it and mixed them properly. The mixture was then refluxed for 2 hr at 140° C. with continuous staring. After the reflux, the solvent was evaporated by evaporator. Then 200 mL of double distilled water, 150 mL diisopropyl ether, and 150 mL H₃PO₄ were added and the total mixture was transferred to a separating funnel. It was then repeatedly washed with double distilled water (3-5 times, 200 mL each time) until the pH of the aqueous phase ˜4. Then the organic phase was collected through sodium phosphate and evaporated to dryness to obtain pure 185 g of Ricinoleic acid (yield 92.5%), confirmed by ¹H NMR.
Synthesis of SUA-RA and DDDA-RA oligomers: SUA-RA and DDDA-RA oligomers were synthesized by esterification reaction of suberic acid and dodecanedioic acid with ricinoleic acid at 170° C. In a round bottom flask, 15 g of SUA, 15 g of RA and catalytic amount (1%) of phosphoric acid were taken and heated to 170° C. for 5 hours under nitrogen. Then another 15 g of RA was added to the round bottom flask and continued to heat for another 4 hours under nitrogen swift. Finally another 5 g of RA was added and again continued to heat over night with mixing under vacuum to yield SUA-RA oligomer with 30:70 ratios of SUA and RA which was characterized by ¹H NMR. DDDA-RA oligomer with 30:70 ratios of DDDA and RA was synthesized following the same procedure and was also characterized by ¹H NMR.
Discussion of the results: Two different oligomers are synthesized using two different dicarboxylic acid and hydroxy acids. RA is esterified with SUA or DDDA under melt and vacuum condition where H₃PO₄ is used as catalyst. Under this reaction condition 100% of the RA is consumed in the esterification reaction with SUA or DDDA which is confirmed from the ¹H NMR as the signal at 3.6 ppm for the alcoholic proton is gone astray after the final step of esterification. Furthermore, self-condensation of RA in this protocol (via step by step addition of RA to SUA or DDDA) is also avoided; evidence form ¹H NMR, as there is no signal at 4.1 ppm. Hence this process gives a well-defined SUA-RA or DDDA-RA oligomers without any residual or self-condensed RA.

### Example 2: Synthesis of Poly(Ester-Anhydride) Approaching from an Alternative Method

The objective is the development an alternative method to synthesis of biodegradable copolymer of poly(ester-anhydride). Here the focus is on two features:
1) Use of sebacic acid (SA) and ricinoleic acid (RA) or 12-hydroxystearic acid (HSA) to prepare SA-RA or SA-HSA oligomers by direct condensation.
2) Use of fewer amounts (1:1 equivalent or less) of acetic anhydride to activate the oligomers for polymerization.
3) Control the molecular weight of poly(ester-anhydride) depending upon amount the acetic anhydride used for the pre-polymerization step.
   Materials: Sebacic acid (SA, 99% pure; Aldrich, USA), 12-hydroxystearic acid (HSA) and acetic anhydride (Merck, Germany) were used as received. Ricinoleic acid (RA) was prepared from the hydrolysis of castor oil as described in the synthesis part.
   Spectral analysis: 1H and 13C NMR spectra were obtained on a Varian 300 MHz NMR spectrometer using CDCl₃ as solvent containing tetramethylsilane as shift reference. Fourier transform infrared (FTIR) spectroscopy was performed using a Smart iTR ATR sampling accessory for Nicolet iS10 spectrometer with a diamond crystal (Thermo Scientific, Massachusetts).
   Molecular weight determination: The molecular weights were determined by gel permeation chromatography (GPC) system, Waters 1515. Isocratic HPLC pump with a Waters 2410 refractive index detector, a Waters 717 plus autosampler, and a Rheodyne (Cotati, CA) injection valve with a 20 μL-loop. The samples were eluted with CHCl₃ (HPLC grade) through linear Styragel HR5 column (Waters) at a flowrate of 1 mL/min. The molecular weights were determined relative to polystyrene standards.
   Synthesis and Characterization: SA-RA oligomers: SA-RA oligomers were synthesized by heating ricinoleic acid and sebacic acid at 175° C. In a round bottom flask, 30 g of SA, 30 g of RA and catalytic amount (0.1%) of phosphoric acid were taken and heated to 170° C. for 5 hours under nitrogen. Then another 30 g of RA was added to the round bottom flask and continued to heat for another 4 hours under nitrogen swift. Finally, another 10 g of RA was added and again continued to heat over night with mixing under vacuum to yield SA-RA oligomer with 30:70 ratios of SA and RA which was characterized by ¹H NMR and FTIR. The SA-RA oligomers of different ratios were also prepared by the same process and characterized by ¹H NMR. The details are given in the Table 1 below.

TABLE 1
SA-RA oligomers

| | | RA | | |
|---|---|---|---|---|
| SA-RA | | 1^{st} Step, 170° C., | 2^{nd} Step 170° C., | 3^{rd} Step 170 ° C., |
| ratio | SA | 5 hrs, N2 | 4 hrs, N2 | Overnight, Vacuum |
| 20:80 | 10 g | 17.5 g | 17.5 g | 5 g |
| 25:75 | 12.5 g | 16.25 g | 16.25 g | 5 g |
| 35:65 | 17.5 g | 13.75 g | 13.75 g | 5 g |

SA-HAS oligomers

SA-HSA oligomers were also synthesized by heating 12-hydroxystearic acid and sebacic acid at 175° C. In a round bottom flask, 15 g of SA, 15 g of HSA and catalytic amount (0.1%) of phosphoric acid were taken and heated to 170° C. for 5 hours under nitrogen. Then another 15 g of HSA was added to the round bottom flask and continued to heat for another 4 hours under nitrogen swift. Finally, another 5 g of HSA was added and again continued to heat over night with mixing under vacuum to yield SA-HSA oligomer with 30:70 ratios of SA and HSA which was characterized by ¹H NMR and FTIR. The SA-HSA oligomers of 20:80 ratios were also prepared by the same process. The details are given in the Table 2 below.

TABLE 2
SA-RA oligomers

| | | HSA | | |
|---|---|---|---|---|
| SA-has | | 1^{st} Step, 170° C., | 2^{nd} Step 170° C., | 3^{rd} Step 170 ° C., |
| ratio | SA | 5 hrs, N2 | 4 hrs, N2 | Overnight, Vacuum |
| 20:80 | 10 g | 17.5 g | 17.5 g | 5 g |

poly(SA-RA)

In a typical synthesis, 10 g of 20:80, 25:75, 30:70, 35:65 ratio of SA-RA oligomers were melt individually at 140° C. under nitrogen atmosphere. Then 1:5 equivalent of acetic anhydride was added to the molten SA-RA oligomers and refluxed at 140° C. for 60 min. Excess acetic anhydride or acetic acid was evaporated. The residue was then subjected to melt condensation at 160° C. under 10 mbar for 4 hours. The SA-RA oligomer of 30:70 ratios was also polymerized under same procedure where different amount (1, 0.7, 0.5, 0.35, 0.25, 0.15 equivalent) of acetic anhydride was used (refluxed at 140° C., overnight) to use fewer amount of acetic anhydride and make a control over the molecular weight.

poly(SA-HSA)

Following the same procedure as poly(SA-RA), 10 g of 20:80 and 30:70 ratio of SA-HSA oligomers were melt individually at 140° C. under nitrogen atmosphere. Then 1:5 equivalent of acetic anhydride was added to both of the molten SA-HSA oligomers and refluxed at 140° C. for 60 min. Excess acetic anhydride or acetic acid was evaporated. The residue was then subjected to melt condensation at 160° C. under vacuum (˜10 m bar) for 4 h.

### Discussion of the Results:

Two kinds of poly(ester-anhydride) copolymers were synthesized through solvent free melt polycondensation process where directly sebacic acid is used to synthesis the SA-RA or SA-HSA oligomers instead of using poly(SA) as starting material. RA or HAS is esterified with SA under melt and vacuum condition where about 1% H₃PO₄ is used as catalyst. Under this reaction condition 100% of the RA or HSA is consumed in the esterification reaction with SA which is confirmed from the ¹H NMR as the signal at 3.6 ppm for the alcoholic proton is gone astray after the final step of esterification. Furthermore, self-condensation of RA or HSA in this protocol (via step by step addition of RA or HAS to SA) is also avoided; evidence form ¹H NMR, as there is no signal at 4.1 ppm. Hence this process gives a well-defined SA-RA or SA-HSA oligomers without any residual or self-condensed RA or HSA. The proton of the esterified polymer chemical shift observed at ˜4.8 ppm. Two protons adjacent to the ester bonds and anhydride bonds arise at 2.43 ppm and 2.33 ppm, respectively.

The molecular weight of the as-synthesized polymers is measured by GPC. The details of the molecular weight and disparity are given in the below Table 3 and control over molecular weight depending upon the acetic anhydride used.

TABLE 3
molecular weight and disparity of polymers of the invention

| | | Molecular | |
|---|---|---|---|
| | | weight | |
| | | (Mw) | polydispersity |
| Sl. No | polymer | Daltons | (PD) |
| 1 | Poly(SA-RA) with 20:80 ratio, using | 17091 | 3.01 |
| | 1:5 w/w acetic anhydride | | |
| 2 | Poly(SA-RA) with 25:75 ratio, using | 18793 | 3.07 |
| | 1:5 w/w acetic anhydride | | |
| 3 | Poly(SA-RA) with 30:70 ratio, using | 12335 | 2.85 |
| | 1:5 w/w acetic anhydride | | |
| 4 | Poly(SA-RA) with 35:65 ratio, using | 18558 | 3.02 |
| | 1:5 w/w acetic anhydride | | |
| 7 | Poly(SA-RA) with 30:70 ratio, using | 4841 | 1.72 |
| | 0.5 equivalent acetic anhydride | | |
| 8 | Poly(SA-RA) with 30:70 ratio, using | 3296 | 1.51 |
| | 0.35 equivalent acetic anhydride | | |
| 9 | Poly(SA-RA) with 30:70 ratio, using | 2357 | 1.35 |
| | 0.25 equivalent acetic anhydride | | |
| 10 | Poly(SA-RA) with 30:70 ratio, using | 1856 | 1.24 |
| | 0.15 equivalent acetic anhydride | | |
| 11 | Poly(SA-HSA) with 20:80 | 15498 | 3.18 |
| | ratio, using 1:5 w/w acetic anhydride | | |
| 12 | Poly(SA-HSA) with 30:70 | 17630 | 3.33 |
| | ratio, using 1:5 w/w acetic anhydride | | |

### Example 3: Synthesis of Poly(SA-RA) with Reduced Reaction Time

Aim: The aim of the project is to monitor the synthesis process via ¹H NMR of biodegradable copolymer of poly(sebacic acid-ricinoleic acid) to reduce the reaction time. Materials: Sebacic acid (SA, 99% pure; Aldrich, USA) was used as received. Ricinoleic acid (RA) was prepared from the hydrolysis of castor oil as described in the synthesis part.

Spectral analysis: ¹H NMR spectra were obtained on a Varian 300 MHz NMR spectrometer using CDCl₃ as solvent. Fourier transform infrared (FTIR) spectroscopy was performed using a Smart iTR ATR sampling accessory for Nicolet iS10 spectrometer with a diamond crystal (Thermo Scientific, Massachusetts).
Molecular weight determination: The molecular weights were determined by gel permeation chromatography (GPC) system, Waters 1515. Isocratic HPLC pump with a Waters 2410 refractive index detector, a Waters 717 plus autosampler, and a Rheodyne (Cotati, CA) injection valve with a 20 μL-loop. The samples were eluted with CHCl₃ (HPLC grade) through
linear Styragel HR5 column (Waters) at a flowrate of 1 mL/min. The molecular weights were determined relative to polystyrene standards.
   Synthesis of SA-RA oligomer: SA-RA oligomers were synthesized by heating ricinoleic acid and sebacic acid at 170° C. In a round bottom flask, 15 g of SA, 15 g of RA and catalytic amount (0.1%) of phosphoric acid were taken and heated to 170° C. for 2 hours under nitrogen. Then another 15 g of RA was added to the round bottom flask and continued to heat for another 2 hours under vacuum for 15 min followed by nitrogen swift. Finally, 5 g of RA was added and again continued to heat for another 8 hours under vacuum to yield SA-RA oligomer with 30:70 w/w ratio of SA and RA which was characterized by ¹H NMR.
   poly(SA-RA): In a typical synthesis, 10 g of SA-RA oligomer with 30:70 ratios were melted at 140° C. under nitrogen atmosphere. Then 1 equivalent of acetic anhydride with respect to the acid in the oligomer was added to the molten SA-RA oligomer and refluxed at 140° C. for 2 hours. Excess acetic anhydride or acetic acid was evaporated. The residue was then subjected to melt condensation at 160° C. under vacuum (˜10 m bar) for 4 hours.

### Discussion of the Results:

RA is esterified with SA under melt and vacuum condition where H₃PO₄ is used as catalyst. Under this reaction condition 100% of the RA is consumed within 12 hours in the esterification reaction with SA. This is confirmed by ¹H NMR, thus, as the signal at 3.6 ppm for the alcoholic proton is gone astray after the final step of esterification. Furthermore, self-condensation of RA in this protocol (via step by step addition of RA to SA) is also avoided; evidence form ¹H NMR, as there is no signal at 4.1 ppm. Then the oligomer was polymerized by refluxing at 140° C. with 1 equivalent of acetic anhydride for 2 hours followed by heating at 160° C. under vacuum for 4 hours. The molecular weight of the polymer is measured by GPC and compared with the polymer that is synthesized from the same SA-RA oligomer with 30:70 ratios by refluxing at 140° C. with 1 equivalent of acetic anhydride for overnight followed by heating at 160° C. under vacuum for 4 hours. It is noticed that both the process gives almost same molecular weight of the polymers (11500 Daltons).

### Example 4: Synthesis of Polyanhydride with Control Molecular Weight

The aim is to develop a method to synthesize a biodegradable polymer of polyanhydrides with controlled molecular weight focusing on:
Use of less amounts of acetic anhydride to activate the acid group of oligomers for polymerization.
Controlling the molecular weight of polyanhydrides.
Determining the effect of a catalyst on polymerization when accessed using various catalysts such as toluene, CaO, ZnO and CaCO3.
   Materials: Suberic acid (C₈H₁₄O₄), Azelaic acid (C₉H₁₆O₄), Sebacic acid (C₁₀H₁₈O₄), Dodecanedioic acid (C₁₂H₂₂O₄), and acetic anhydride were used as received. Spectral analysis: Fourier transform infrared (FTIR) spectroscopy was performed using a Smart iTR ATR sampling accessory for Nicolet iS10 spectrometer with a diamond crystal (Thermo Scientific, Massachusetts).
   Molecular weight determination: Molecular weights were determined by gel permeation chromatography (GPC) system, Waters 1515. Isocratic HPLC pump with a Waters 2410 refractive index detector, a Waters 717 plus autosampler, and a Rheodyne (Cotati, CA) injection valve with a 20 μL-loop. The samples were eluted with CHCl₃ (HPLC grade) through linear Styragel HR5 column (Waters) at a flowrate of 1 mL/min. The molecular weights were determined relative to polystyrene standards.
   Synthesis of different polyanhydrides: In a typical synthesis, 2 g of each diacid monome (Suberic acid, Azelaic acid, Sebacic acid, and Dodecanedioic acid) were melted at 140° C. under a nitrogen atmosphere. Then different amounts (5 equiv., 1.0 equiv., 0.5 equiv., or 0.25 equiv.) of acetic anhydride with respect to carboxylic acid groups were added to the every molten diacid monomer and refluxed at 140° C. for 1 hr. Any excess acetic anhydride or acetic acid was evaporated. The residue was then subjected to melt condensation at 160° C. under vacuum (˜10 m bar) for 4 h and characterized by NMR, FTIR, and GPC.

Poly(sebacic acid) was also synthesized by heating sebacic acid with acetic anhydride (0.3 equiv.) and other catalysts in a closed system followed by the polymerization through melt condensation. The sebacic acid (25 g, 123.6 mmol, 1 equiv.) was activated by overnight heating at 160° C. in a closed condition with acetic anhydride (7.0 mL, 74.1 mmol, 0.3 equiv.). Five different types of polymerization were performed using activated sebacic acid (2.0 g, 9.9 mmol) with different catalysts such as toluene (2.0 mL, 1:1 w/v), CuO (5.6 mg, 0.099 mmol, 1 mol %), ZnO (8.1 mg, 0.099 mmol, 1 mol %) or CuCO₃ (9.9 mg, 0.099 mmol, 1 mol %), and neat condition (without any catalyst) as a control experiment. Each reaction mixture was polymerized by melt condensation at 160° C. for 4 h under vacuum (10 mbar) with constant stirring. The polymerization was monitored by NMR, GPC, and FT-IR.

Results: A series of polyanhydrides is synthesized through a solvent-free melt polycondensation process from different dicarboxylic acids. The FTIR spectra for polysuberic acid, polyazelaic acid, and polydodecanedioic acid, respective of all the as-synthesized polyanhydrides, shows that the C═Ostretching frequency of acid group and anhydride group in every case arises at ˜1700 cm⁻¹ and ˜1815 cm⁻¹. The ratios of peak height of anhydride with respect to acid shows a gradual increase from 0.25 equivalents to 1 or 5 equivalents of used acetic anhydride.

Polymerization was monitored by FT-IR by investigating the presence of carboxylic acid and anhydride functionalities. When 0.25 equiv. of acetic anhydride is used, the weak frequencies at 1810 cm⁻¹ and 1740 cm⁻¹, and the strong frequency around 1700 cm⁻¹, correspond to anhydride and carboxylic acid bonds respectively, indicating the presence of acids and partial conversion to anhydrides due to the formation of polyanhydrides with low molecular weight. When increasing the quantity of acetic anhydride from 0.25 equiv. to 1.0 equiv., an increase in the anhydride bond and a decrease in the acid bond were observed. When 1 equiv. of acetic anhydride is used, almost all the acids are converted into anhydrides.

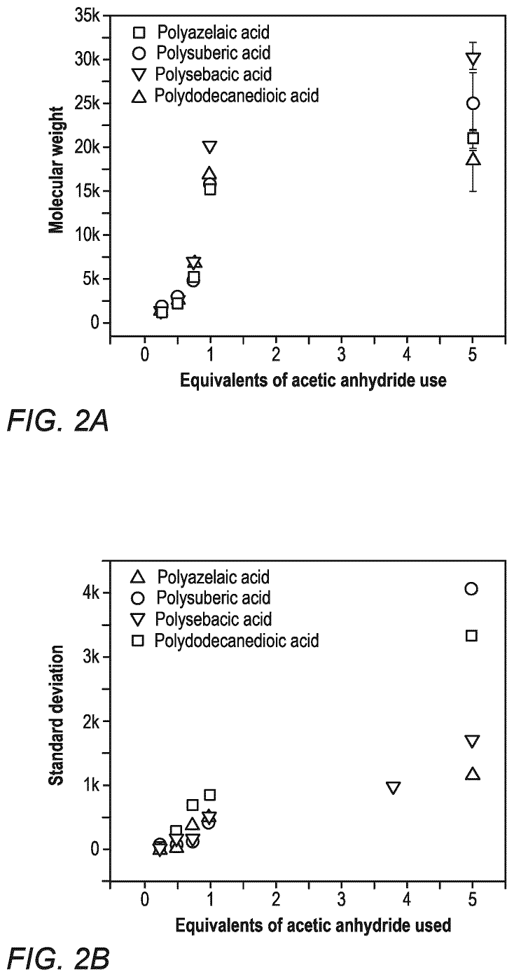

After polymerization by melt condensation at 16° C. for 4 h under vacuum (10 mbar), the molecular weight was analysed by GPC. The analysis revealed that the molecular weight of each polyanhydride gradually increases when adding more acetic anhydride from 0.25 equiv. to 1 or 5equiv. The study revealed that 1.0 equiv. of acetic anhydride is enough to get almost similar molecular weight when 5 equiv. (excess) acetic anhydride was used. FIG. 2 is a diagram exemplifying control over the molecular weight of polyanhydride depending upon the acetic anhydride used, wherein the variation of molecular weight is plotted against the amount of acetic anhydride used during the activation of monomer, FIG. 2A. FIG. 2B depicts a variation of standard deviation of the molecular weights of the synthesized polyanhydrides (in triplex) depending upon the acetic anhydride used to activate the monomers. The molecular weight and standard deviation of polyanhydride increases with the increasing amount of acetic anhydride for the activation of diacids toward polymerization.

Conclusion: An effective route for the synthesis of aliphatic polyanhydride made from a series of dicarboxylic acids with control molecular weight is reported. The molecular weights of the synthesized polymers in this reported protocol are highly controllable depending upon the degree of activation of the monomers. This route forwarded an idea for producing aliphatic polyanhydride with control molecular weight for possible use in the preparation of degradable disposable medical supplies.

### Example 5: Synthesis of Stable Polyanhydrides from the Polycondensation of Dicarboxylic Acid Monomers Having a Fatty Side Chain

In P(SA-RA) the alternating architecture and hydrophobic side chains hinder the hydrolytic cleavage and anhydride interchange. A series of polyanhydrides was synthesised to investigate the effect of ester bonds, hydrophobic side chains, phenyl moieties, distance from anhydride bonds on their stability, and other properties. Hydroxy acid is converted to ester-diacid by the esterification reaction with anhydrides. Polyanhydrides were obtained by the activation of ester-diacid using acetic anhydride followed by melt condensation. The reactions were monitored by NMR, FT-IR and GPC. The synthesised poly(ester-anhydride)s with a shorter chain length compared to P(SA-RA) were stable at room temperature. Hydrolytic degradation studies revealed that the phenyl moiety present in PRAP and PHSAP reduces the hydrolysis of anhydride bonds. The results reveal that the presence of hydrophobic side chains and their distance from anhydride bonds significantly improve shelf life stability.

### Experimental

### Materials

Hydroxy acids such as 12-hydroxydodecanoic acid (HDDA, 97%, Aldrich), 2-hydroxyoctanoic acid (HOA, 98%, Alfa Aesar) and 12-hydroxystearic acid (HSA, 75%, TCI) were used as received. Diacids such as sebacic acid (SA, 99%) and dodecanedioic acid (DDDA, 99%) were purchased form Sigma-Aldrich and used as received. Anhydrides such as succinic anhydride (99%; Aldrich), maleic anhydride (99%; Sigma-Aldrich), phthalic anhydride (99%; Aldrich) and acetic anhydride (Merck) were purchased and used as obtained. Castor oil was purchased from Tamar (Jerusalem, Israel). All solvents and reagents (analytical-grade) were purchased (Sigma-Aldrich or BioLab) and used without further purification.

### Spectral Analysis

Fourier transform infrared (FTIR) spectroscopy was performed using a Smart iTR ATR sampling accessory for Nicolet iS10 spectrometer with a diamond crystal (Thermo Scientific, Massachusetts). 1H and ¹³C NMR spectra were obtained on a Varian 300 MHz and 75 MHz NMR spectrometer respectively, in tubes with 5 mm outside diameters. CDCl₃ or DMSO-d₆ served as a solvent. Thin layer chromatography (TLC) plates are purchased from Merck (Silica gel matrix coated with a flourescent indicator on aluminium plates).

### Molecular Weight Determination

Molecular weights were determined by gel permeation chromatography (GPC) system, Waters 1515. Isocratic HPLC pump with a Waters 2410 refractive index detector, a Waters 717 plus autosampler, and a Rheodyne (Cotati, CA) injection valve with a 20 μL-loop. The samples were eluted with CHCl₃ (HPLC grade) through a linear Styragel HR4E column (Waters) with a molecular-weight range of 50-100K Da at a flowrate of 1 mL/min. The molecular weights were determined relative to polystyrene standards (Polyscience, Warrington, PA).

### Synthesis of Polyanhydrides

### Ricinoleic Acid (RA)

Ricinoleic acid (RA) was prepared from the hydrolysis of castor oil as previously described. 3 Castor oil (200 g) was hydrolysed by refluxing in a KOH (48 g) solution (ethanol, 400 mL) for 2 h. After the evaporation of ethanol, DDW (400 mL) was added to the reaction flask. The clear yellowish solution was acidified with H₃PO₄ to reach pH ˜2. The obtained fatty acid was extracted with diisopropyl ether. The organic layer was dried over anhydrous Na₂SO₄, filtered, and evaporated to dryness. RA was obtained as a pale-yellow clear viscous liquid. 12-Hydroxyoctadec-9-enoic acid; ¹H NMR (300 MHz, Chloroform-d) δ 5.56 (dt, J=10.8, 7.4 Hz, 1H), 5.47-5.30 (m, 1H), 3.62 (p, J=6.1 Hz, 1H), 2.34 (t, J=7.4 Hz, 2H), 2.21 (t, J=6.9 Hz, 2H), 2.04 (q, J=6.8 Hz, 2H), 1.63 (p, J=7.2 Hz, 2H), 1.48-1.43 (m, 2H), 1.37— 1.20 (m, 16H), 0.88 (t, J=6.0 Hz, 3H); FTIR (cm ¹) 3008, 2924, 2854, 1708, 1457, 1410, 1244.

Poly(sebacic acid) (PSA)

PSA was synthesized by reflux of sebacic acid with acetic anhydride (1:5w/v) followed by the polymerisation through melt condensation. PSA was synthesized by refluxing sebacic acid (50 g) with acetic anhydride (250 mL, 1:5w/v) for 30 min with constant stirring. The excess acetic anhydride was evaporated to dryness under vacuum. The clear residue was further polymerized by melt condensation at 160° C. for 4 h under vacuum (10 mbar) with constant stirring. PSA was obtained as a pale yellow solid.

Poly(dodecanedioic acid) (PDDDA)

PDDA was synthesized by reflux of dodecanedioic acid with acetic anhydride (1:5w/v), followed by the polymerisation through melt condensation. PDDDA was synthesized by refluxing dodecanedioic acid (50 g) with acetic anhydride (250 mL, 1:5w/v) for 30 min with constant stirring. The excess acetic anhydride was evaporated to dryness under vacuum. The clear residue was further polymerized by melt condensation at 160° C. for 4 h under vacuum (10 mbar) with constant stirring. PDDDA was obtained as a pale yellow solid.

### Poly(Sebacic Acid-Ricinoleic Acid) (P(SA-RA))

P(SA-RA) was synthesized using PSA and RA with 30% and 70% weight ratios respectively. PSA (15 g) and RA (35 g) were melted and stirred at 175° C. under inert nitrogen atmosphere. The molten mixture was kept for 24 h under inert atmosphere until no free RA remained in the reaction mixture. After 24 h, acetic anhydride (250 mL, 1:5w/v) was added and refluxed at 140° C. for 30 min. Excess acetic anhydride was evaporated under vacuum at 70° C. The residue was then subjected to melt condensation at 160° C. under vacuum (10 m bar) for 6 h. P(SA-RA) was obtained as a pale-yellow clear pasty polymer. ¹H NMR (300 MHz, CDCl₃) δ 5.54-5.40 (m, 1H), 5.40-5.26 (m, 1H), 4.88 (p, J=6.3 Hz, 1H), 2.45 (t, J=7.4 Hz, 2H), 2.32-2.21 (m, 4H), 2.02 (q, J=7.7, 7.0 Hz, 2H), 1.77-1.44 (m, 8H), 1.37-1.21 (m, 26H), 0.87 (t, J=6.0 Hz, 3H).

### Poly(Sebacic Acid-Hydroxystearic Acid) (P(SA-HSA))

P(SA-HSA) was synthesized using PSA and HSA with 30% and 70% weight ratios respectively. PSA (15 g) and HSA (35 g) were melted and stirred at 175° C. under inert nitrogen atmosphere. The molten mixture was kept for 24 h under inert atmosphere until no free HSA remains in the reaction mixture. After 24 h, acetic anhydride (250 mL, 1:5w/v) was added and refluxed at 140° C. for 30 min. Excess acetic anhydride was evaporated under vacuum at 70° C. The residue was then subjected to melt condensation at 160° C. under vacuum (10 m bar) for 6 h. P(SA-HSA) was obtained as a pale-yellow clear pasty polymer. ¹H NMR (300 MHz, CDCl₃) δ 4.86 (p, J=6.2 Hz, 1H), 2.34 (t, J=7.4 Hz, 2H), 2.27 (t, J=7.5 Hz, 2H), 1.61 (q, J=7.0 Hz, 6H), 1.52-1.43 (m, 4H), 1.32-1.22 (m, 32H), 0.87 (t, J=5.8 Hz, 3H).

### Ricinoleic Acid Succinate (RAS)

RAS was prepared by a previously reported method with modifications.⁵ A solution of RA (20.0 g, 67 mmol, 1.0 equiv.) and succinic anhydride (8.1 g, 80 mmol, 1.2 equiv.) in toluene (80 mL) was stirred at 90° C. The reaction was monitored by TLC using hexane/ethyl acetate/acetic acid (80/30/1 v/v/v) as an eluent, and a vanillin stain was used to identify the spots. After the full conversion of RA, the reaction mixture was cooled to RT, and toluene was removed using a rotoevaporater. Then, water was added to the residue and stirred for 15 min. Subsequently, ethyl acetate was used for the extraction, and the organic layer was washed three times with distilled water. Then, the organic layer was dried over anhydrous Na₂SO₄ and evaporated to dryness. RAS was obtained with 96% yield (25.7 g) as a pale yellow viscus liquid, which solidified to white solid at RT. 12-((3-Carboxypropanoyl)oxy)octadec-9-enoic acid; ¹H NMR (300 MHz, CDCl₃) δ 5.55-5.39 (m, 1H), 5.39-5.23 (m, 1H), 4.90 (p, J=6.3 Hz, 1H), 2.67 (t, J=6.1 Hz, 2H), 2.61 (t, J=5.8 Hz, 2H), 2.35 (t, J=7.4 Hz, 2H), 2.31-2.24 (m, 2H), 2.01 (q, J=7.2 Hz, 2H), 1.71-1.59 (m, 2H), 1.59-1.46 (m, 2H), 1.35-1.23 (m, 16H), 0.87 (t, J=6.0 Hz, 3H); FTIR (cm⁻¹) 3008, 2925, 2855, 1732, 1707, 1458, 1411, 1169.

### Poly(Ricinoleic Acid Succinate) (PRAS)

PRAS was synthesized by reflux of RAS with acetic anhydride followed by polymerisation through melt condensation. PRAS was synthesized by refluxing RAS (10 g) with acetic anhydride (50 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PRAS was obtained as a pale-yellow clear pasty polymer. Weight-average MW by GPC=14654 (PDI=1.83); ¹H NMR (300 MH, CDCl₃) δ 5.54-5.40 (m, 1H), 5.39-5.23 (m, 1H), 4.89 (p, J=6.3 Hz, 1H), 2.75 (t, J=6.7 Hz, 2H), 2.63 (t, J=7.1 Hz, 2H), 2.44 (q, J=7.1 Hz, 2H), 2.35-2.21 (m, 2H), 2.01 (q, J=6.5 Hz, 2H), 1.66 (p, J=7.3 Hz, 2H), 1.59-1.45 (m, 2H), 1.32-1.23 (m, 16H), 0.87 (t, J=6.0 Hz, 3H); FTIR (cm⁻¹) 3010, 2925, 2855, 1819, 1732, 1463, 1410, 1182, 1037.

### Ricinoleic Acid Maleate (RAM)

A solution of RA (20.0 g, 67 mmol, 1.0 equiv.) and maleic anhydride (7.9 g, 80 mmol, 1.2 equiv.) in toluene (80 mL) was stirred at 90° C. The reaction was monitored by TLC using hexane/ethyl acetate/acetic acid (80/30/1 v/v/v) as an an eluent, and vanillin stain was used to identify the spots. The reaction mixture was cooled to RT after the full conversion of RA and toluene was removed using a rotoevaporater. Then, water was added to the residue and stirred for 15 min at 50° C. Ethyl acetate was used for the extraction, and the organic layer was washed three times with distilled water. Then, the organic layer was dried over anhydrous Na₂SO₄ and evaporated to dryness. RAM was obtained with 94% yield (25.0 g) as a pale orange viscus liquid. 12-((−3-Carboxyacryloyl)oxy)octadec-9-enoic acid; ¹H NMR (300 MHz, CDCl₃) δ 11.41 (s, 2H), 6.39 (d, J=12.5 Hz, 1H), 6.34 (d, J=12.5 Hz, 1H), 5.59-5.41 (m, 1H), 5.40-5.24 (m, 1H), 5.02 (p, J=6.3 Hz, 1H), 2.36 (t, J=7.4 Hz, 4H), 2.02 (q, J=7.3 Hz, 2H), 1.62 (q, J=7.0 Hz, 4H), 1.33-1.25 (m, 16H), 0.88 (t, J=6.0 Hz, 3H); FTIR (cm⁻¹) 3011, 2925, 2855, 1705, 1645, 1411, 1247, 1214, 1168.

### Poly(Ricinoleic Acid Maleate) (PRAM)

PRAM was synthesized by reflux of RAM with acetic anhydride followed by polymerisation through melt condensation. PRAM was synthesized by refluxing RAM (10 g) with acetic anhydride (50 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PRAS was obtained as a pale-brown colour clear pasty polymer. Weight-average MW by GPC=11876 (PDI=1.87); ¹H NMR (300 MHz, CDCl₃) δ 7.05-6.73 (m, 2H), 5.58-5.40 (m, 1H), 5.35-5.27 (m, 1H), 4.99 (p, J=6.4 Hz, 1H), 2.54-2.41 (m, 2H), 2.36-2.26 (m, 2H), 2.01 (q, J=7.7 Hz, 2H), 1.68-1.57 (m, 4H), 1.31-1.24 (m, 16H), 0.88 (t, J=5.8 Hz, 3H); FTIR (cm⁻¹) 3011, 2925, 2855, 1815, 1723, 1643, 1464, 1287, 1259, 1179, 1040.

### Ricinoleic Acid Phthalate (RAP)

A solution of RA (20.0 g, 67 mmol, 1.0 equiv.) and phthalic anhydride (11.9 g, 80 mmol, 1.2 equiv.) in toluene (80 mL) was stirred at 90° C. The reaction was monitored by TLC using hexane/ethyl acetate/acetic acid (80/30/1 v/v/v) as an eluent, and vanillin stain was used to identify the spots. After the full conversion of RA, the reaction mixture was cooled to RT, and toluene was removed using an evaporator. Then, water was added to the residue and stirred for 15 min at 50° C.Ethyl acetate was used for the extraction, and the organic layer was washed three times with distilled water. The organic layer was dried over anhydrous Na₂SO₄ and evaporated to dryness. RAP was obtained with 88% yield (26.3 g) as a pale orange viscus liquid. 2-(((17-Carboxyheptadec-9-en-7-yl)oxy)carbonyl)benzoic acid; ¹H NMR (300 MHz, CDCl₃) δ 7.84 (dd, J=6.6, 2.3 Hz, 1H), 7.72 (dd, J=6.9, 2.0 Hz, 1H), 7.58 (dt, J=7.6, 5.9 Hz, 2H), 5.61-5.44 (m, 1H), 5.42-5.30 (m, 1H), 5.11 (p, J=6.3 Hz, 1H), 2.47-2.28 (m, 4H), 2.08-2.00 (m, 2H), 1.64 (p, J=5.8, 4.8 Hz, 4H), 1.34-1.25 (m, 16H), 0.86 (t, J=6.3 Hz, 3H); FTIR (cm⁻¹) 3009, 2925, 2854, 2667, 1701, 1600, 1580, 1455, 1411, 1284, 1125, 1073.

### Poly(Ricinoleic Acid Phthalate) (PRAP)

PRAP was synthesized by reflux of RAP with acetic anhydride followed by polymerisation through melt condensation. PRAP was synthesized by refluxing RAP (10 g) with acetic anhydride (50 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PRAS was obtained as a pale-brown clear pasty polymer. Weight-average MW by GPC=8391 (PDI=1.86); ¹H NMR (300 MHz, CDCl₃) δ 7.84-7.74 (m, 1H), 7.74-7.65 (m, 1H), 7.63-7.48 (m, 2H), 5.46-5.40 (m, 1H), 5.34-5.28 (m, 1H), 4.87 (p, J=6.4 Hz, 1H), 2.46-2.40 (m, 2H), 2.28-2.24 (m, 2H), 2.02-2.00 (m, 2H), 1.63-1.53 (m, 4H), 1.28-1.25 (m, 16H), 0.86 (t, J=6.0 Hz, 3H); FTIR (cm⁻¹) 3010, 2925, 2854, 1814, 1727, 1598, 1579, 1464, 1410, 1281, 1209, 1132, 1090, 1014.

### Hydroxystearic Acid Succinate (HSAS)

A solution of 12-hydroxystearic acid (HAS) (20.0 g, 67 mmol, 1.0 equiv.) and succinic anhydride (8.0 g, 80 mmol, 1.2 equiv.) in toluene (80 mL) was stirred at 90° C. The reaction was monitored by TLC using hexane/ethyl acetate/acetic acid (80/30/1 v/v/v) as an eluent, and vanillin stain was used to identify the spots. After the full conversion of HSA, the reaction mixture was cooled to RT, and toluene was removed using a rotoevaporater. Then, water was added to the residue and stirred for 15 min. Ethyl acetate was used for extraction, and the organic layer was washed three times with distilled water. Then, the organic layer was dried over anhydrous Na₂SO₄ and evaporated to dryness. HSAS was obtained with 95% yield (25.4 g) as a white solid. 12-((3-Carboxypropanoyl)oxy)octadecanoic acid; ¹H NMR (300 MHz, CDCl₃) δ 4.90 (p, J=6.3 Hz, 1H), 2.74-2.65 (m, 2H), 2.65-2.57 (m, 2H), 2.34 (t, J=7.2 Hz, 2H), 1.63 (q, J=7.1 Hz, 2H), 1.59-1.41 (m, 6H), 1.28-1.25 (m, 20H), 0.88 (t, J=6.0 Hz, 3H); FTIR (cm⁻¹) 2922, 2853, 1708, 1466, 1411, 1380, 1343, 1288, 1170.

### Poly(Hydroxystearic Acid Succinate) (PHSAS)

PHSAS was synthesized by reflux of HSAS with acetic anhydride followed by polymerisation through melt condensation. PHSAS was synthesized by refluxing HSAS (10 g) with acetic anhydride (50 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PHSAS was obtained as a pale-yellow colour clear pasty polymer. Weight-average MW by GPC=19081 (PDI=2.44); ¹H NMR (300 MHz, CDCl₃) δ 4.88 (p, J=6.3 Hz, 1H), 2.77 (t, J=6.7 Hz, 2H), 2.65 (t, J=6.4 Hz, 2H), 2.51-2.42 (m, 2H), 1.65 (p, J=7.2 Hz, 2H), 1.52 (q, J=6.5 Hz, 6H), 1.31-1.25 (m, 20H), 0.88 (t, J=6.4 Hz, 3H); FTIR (cm⁻¹) 2925, 2854, 1820, 1732, 1465, 1411, 1378, 1356, 1184, 1040.

### Hydroxystearic Acid Maleate (HSAM)

A solution of HSA (20.0 g, 67 mmol, 1.0 equiv.) and maleic anhydride (7.8 g, 80 mmol, 1.2 equiv.) in toluene (80 mL) was stirred at 90° C. The reaction was monitored by TLC using hexane/ethyl acetate/acetic acid (80/30/1 v/v/v) as an eluent, and vanillin stain was used to identify the spots. After the full conversion of HSA, the reaction mixture was cooled to RT, and toluene was removed using a rotoevaporater. Then, water was added to the residue and stirred for 15 min at 50° C. Ethyl acetate was used for extraction, and the organic layer was washed three times with distilled water. Then, the organic layer was dried over anhydrous Na₂SO₄ and evaporated to dryness. HSAM was obtained with 92% yield (24.5 g) as a white solid. 12-((3-Carboxyacryloyl)oxy)octadecanoic acid; ¹H NMR (300 MHz, CDCl₃) δ 6.40 (d, J=12.0 Hz, 1H), 6.35 (d, J=12.0 Hz, 1H), 5.01 (p, J=6.2 Hz, 1H), 2.34 (t, J=7.3 Hz, 2H), 1.67-1.54 (m, 6H), 1.31-1.23 (m, 22H), 0.87 (t, J=6.0 Hz, 3H); FTIR (cm⁻¹) 3012, 2924, 2854, 1704, 1645, 1456, 1411, 1379, 1216, 1170.

### Poly(Hydroxystearic Acid Maleate) (PHSAM)

PHSAM was synthesized by reflux of HSAM with acetic anhydride followed by polymerisation through melt condensation. PHSAM was synthesized by refluxing HSAM (10 g) with acetic anhydride (50 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PHSAM was obtained as a pale-brown clear pasty polymer. Weight-average MW by GPC=23613 (PDI=2.69); ¹H NMR (300 MHz, CDCl₃) δ 6.96-6.82 (m, 1H), 6.36-6.25 (m, 1H), 4.98 (p, J=5.9 Hz, 1H), 2.57-2.38 (m, 2H), 1.71-1.52 (m, 6H), 1.43-1.24 (m, 22H), 0.87 (t, J=6.0 Hz, 3H); FTIR (cm⁻¹) 3012, 2924, 2854, 1815, 1720, 1640, 1464, 1394, 1284, 1223, 1181, 1037.

### Hydroxystearic Acid Phthalate (HSAP)

A solution of HSA (20.0 g, 67 mmol, 1.0 equiv.) and phthalic anhydride (11.8 g, 80 mmol, 1.2 equiv.) in toluene (80 mL) was stirred at 90° C. The reaction was monitored by TLC using hexane/ethyl acetate/acetic acid (80/30/1 v/v/v) as an eluent, and vanillin stain was used to identify the spots. After the full conversion of HSA, the reaction mixture was cooled to RT, and toluene was removed using a evaporator. Then, water was added to the residue and stirred for 15 min at 50° C. Ethyl acetate was used for the extraction, and the organic layer was washed three times with distilled water. Then, the organic layer was dried over anhydrous Na₂SO₄ and evaporated to dryness. HSAP was obtained with 90% yield (26.8 g) as a white solid. 2-(((17-Carboxyheptadecan-7-yl)oxy)carbonyl)benzoic acid; 1H NMR (300 MHz, CDCl₃) δ 7.88 (d, J=7.3 Hz, 1H), 7.66 (d, J=7.4 Hz, 1H), 7.63-7.46 (m, 2H), 5.13 (p, J=6.2 Hz, 1H), 2.36 (t, J=7.3 Hz, 2H), 1.77-1.54 (m, 6H), 1.49-1.25 (m, 22H), 0.85 (t, J=6.8 Hz, 3H); FTIR (cm 1) 3010, 2924, 2854, 1699, 1600, 1580, 1491, 1455, 1411, 1283, 1126, 1073.

### Poly(Hydroxystearic Acid Phthalate) (PHSAP)

PHSAP was synthesized by reflux of HSAP with acetic anhydride followed by polymerisation through melt condensation. PHSAP was synthesized by refluxing HSAP (10 g) with acetic anhydride (50 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PHSAP was obtained as a dark-brown colour clear pasty polymer. Weight-average MW by GPC=11428 (PDI=1.96); ¹H NMR (300 MHz, CDCl₃) δ 7.82-7.76 (m, 1H), 7.74-7.64 (m, 1H), 7.61-7.54 (m, 2H), 5.08 (p, J=6.1 Hz, 1H), 2.56 (t, J=7.3 Hz, 1H), 2.42 (t, J=7.4 Hz, 1H), 1.72 -1.55 (m, 8H), 1.42-1.26 (m, 20H), 0.86 (t, J=6.0 Hz, 5H); FTIR (cm 1) 3010, 2924, 2854, 1815, 1722, 1598, 1579, 1465, 1407, 1282, 1210, 1133, 1014.

### Hydroxydodecanoic Acid Succinate (HDDAS)

A solution of 12-hydroxydodecanoic acid (HDDA) (5.0 g, 23 mmol, 1.0 equiv.) and succinic anhydride (2.8 g, 28 mmol, 1.2 equiv.) in toluene (25 mL) was stirred at 90° C. The reaction was monitored by TLC using hexane/ethyl acetate/acetic acid (80/30/1 v/v/v) as an eluent, and vanillin stain was used to identify the spots. After the full conversion of HDDA, the reaction mixture was cooled to RT, and toluene was removed using an evaporator. Then, water was added to the residue and stirred for 15 min. Ethyl acetate was used for the extraction, and the organic layer was washed three times with distilled water. Then, the organic layer was dried over anhydrous Na₂SO₄ and evaporated to dryness. HDDAS was obtained with 91% yield (6.7 g) as a white solid. 12-((3-Carboxypropanoyl)oxy)dodecanoic acid; ¹H NMR (300 MHz, CDCl₃) δ 4.12 (t, J=6.4 Hz, 2H), 2.75-2.66 (m, 2H), 2.66-2.57 (m, 2H), 2.36 (t, J=6.9 Hz, 2H), 1.73-1.55 (m, 4H), 1.45-1.19 (m, 14H).

### Poly(Hydroxydodecanoic Acid Succinate) (PHDDAS)

PHDDAS was synthesized by reflux of HDDAS with acetic anhydride followed by polymerisation through melt condensation. PHDDAS was synthesized by refluxing HDDAS (5 g) with acetic anhydride (25 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PHDDAS was obtained as a pale-brown colour solid. Weight-average MW by GPC=7981 (PDI=2.65); ¹H NMR (300 MHz, CDCl₃) δ 4.09 (t, J=6.7 Hz, 2H), 2.77 (t, J=6.9 Hz, 2H), 2.66 (t, J=6.5 Hz, 2H), 2.45 (q, J=7.3 Hz, 2H), 1.75-1.53 (m, 4H), 1.45-1.24 (m, 14H); FTIR (cm⁻¹) 2916, 2849, 1816, 1744, 1464, 1417, 1320, 1184, 1125, 1045.

### Hydroxyoctanoic Acid Succinate (HOAS)

A solution of 2-hydroxyoctanoic acid (HOA) (5.0 g, 31 mmol, 1.0 equiv.) and succinic anhydride (3.8 g, 38 mmol, 1.2 equiv.) in toluene (25 mL) was stirred at 90° C. The reaction was monitored by TLC using hexane/ethyl acetate/acetic acid (80/30/1 v/v/v) as eluent and vanillin stain was used to identify the spots. After the full conversion of HOA, the reaction mixture was cooled to RT, and toluene was removed using an evaporator. Then, water was added to the residue and stirred for 15 min. Ethyl acetate was used for extraction, and the organic layer was washed three times with distilled water. Then, the organic layer was dried over anhydrous Na₂SO₄ and evaporated to dryness. HOAS was obtained with 92% yield (8.1 g) as a white solid. 2-((3-Carboxypropanoyl)oxy)octanoic acid; ¹H NMR (300 MHz, CDCl₃) δ 4.93 (t, J=6.5 Hz, 1H), 2.93-2.77 (m, 2H), 2.65-2.45 (m, 2H), 1.87 (q, J=6.6 Hz, 2H), 1.53-1.38 (m, 2H), 1.38-1.25 (m, 6H), 0.88 (t, J=6.0 Hz, 3H).

### Poly(Hydroxyoctanoic Acid Succinate) (PHOAS)

PHOAS was synthesized by reflux of HOAS with acetic anhydride followed by polymerisation through melt condensation. PHOAS was synthesized by refluxing HOAS (5 g) with acetic anhydride (25 mL, 1:5w/v) for 30 min with constant stirring. The excess acetic anhydride was evaporated to dryness under vacuum at 70° C. The clear residue was further polymerized by melt condensation at 140° C. for 6 h under vacuum (10 mbar) with constant stirring. PHOAS was obtained as a dark-brown colour pasty polymer. ¹H NMR (300 MHz, CDCl₃) δ 5.07 (t, J=6.5 Hz, 1H), 4.50-3.78 (m, 2H), 2.88-2.57 (m, 2H), 2.05-1.72 (m, 2H), 1.55-1.03 (m, 8H), 0.88 (t, J=6.0 Hz, 3H); FTIR (cm⁻¹) 2955, 2927, 2860, 1827, 1747, 1458, 1378, 1360, 1170, 1062, 1033.

### Stability Studies

### Storage Stability

The polyanhydrides were investigated for their storage stability at room temperature. All the samples (˜50 mg, in duplicate) were kept at room temperature (˜25° C.) in a nitrogen atmosphere. The change in the molecular weight was regularly recorded for 3 months using GPC and the results were compared with PSA and P(SA-RA).

### Hydrolytic Degradation Studies

Twelve polymer samples (˜100 mg, in duplicate), e.g., PSA, PDDA, P(SA-RA), P(SA-HAS), PRAS, PRAM, PRAP, PHSAS, PHSAM, PHSAP, PHOAS and PHDDAS were analyzed for hydrolytic degradation. Each sample was taken in a 1 mL Eppendorf tube containing 1 mL of a 0.1 M phosphate buffer solution (PBS, pH 7.4). Then, all the samples were kept at 37° C. with constant shaking (100 rpm). Totally five independent sample sets were used to study hydrolysis at different intervals (1 day, 3 days, 7 days, 14 days, and 30 days). The buffer was replaced at regular intervals. Each time point (after 1 day, 3 days, 7 days, 14 days, and 30 days), the buffer was removed from polymer samples and lyophilized. The hydrolysis was monitored and compared with the initial polymers by FT-IR spectroscopy and molecular weight by GPC.

### Results and Discussions

### Design and Synthesis

Various polyanhydrides were designed to investigate the effect of ester bonds, hydrophobic side chains, phenyl moieties, and their distance from anhydride bonds on their stability and properties. PDDDA and P(SA-HSA) (30:70) were used instead of PSA and P(SA-HSA) (30:70) to keep the same length in the polymeric backbone chain. PHDDAS was designed to evaluate the effect of ester bonds in poly(ester-anhydride) compare to only polyanhydride (PDDDA). PHSAS was designed to investigate the effect of decreasing the polymeric backbone chain length, thereby making hydrophobic side chains closer to anhydride bonds. In PHSAP, phenyl moieties were incorporated into hydrophobic side chains to study their properties. Finally, PHOAS was designed to reduce the polymeric backbone chain length and to make hydrophobic side chains very close to anhydride bonds.

The detailed synthetic methodology is given in Scheme 1b. In the first step, hydroxy acid is converted to ester-diacid by the esterification reaction with anhydrides using toluene as a solvent at 90° C. Then, the ester-diacid is activated using acetic anhydride. Finally, the poly(ester-anhydrides) are obtained by melt condensation. Synthesis of ester-diacid was optimised using RA and succinic-, maleic- and phthalic anhydrides. RA was reacted with an excess quantity of anhydrides at 90° C. in toluene for the complete conversion of RA to avoid purification. If the excess amount of anhydride was taken, it had to be removed by the washing with water. However, only succinic anhydride is highly reactive with water. Maleic and phthalic anhydrides are less reactive with water. Thus, anhydrides were removed by the heating with water at 50° C. for 30 min after the complete consumption of RA. Before the addition of water, toluene was removed to avoid the formation of an emulsion. The reaction progress was monitored by TLC using vanillin stain. Ester-diacids such as RAS, RAM and RAP were obtained as a viscous liquid.

Subsequently, this protocol extended to other hydroxy acids. HSA was reacted with succinic-, maleic- and phthalic anhydride and obtained ester-diacids such as HSAS, HSAM and HSAP, respectively, as solids. HOA and HDDA were reacted with succinic anhydride and obtained ester-diacids such as HOAS (liquid) and HDDAS (solid), respectively. After the synthesis of all monomers (RAS, RAM, RAP, HSAS, HSAM, HSAP, HOAS and HDDAS), the synthesis of poly(ester-anhydride) such as PRAS, PRAM, PRAP, PHSAS, PHSAM, PHSAP, PHOAS and PHDDAS was performed by melt condensation. First, the ester-diacid monomers were activated through the reflux with 1:5w/v acetic anhydride for 30 min. Excess acetic anhydride was evaporated to dryness under vacuum at 70° C. The clear residue was further polymerized by melt condensation at 140° C. for 6 h under vacuum (10 mbar) with constant stirring, which provided poly(ester-anhydride)s as the pasty polymer.

PSA, PDDDA, P(SA-RA) and P(SA-HSA) were prepared to compare the stability and properties of the newly designed and synthesized poly(ester-anhydride)s. PSA and PDDDA were prepared using SA and DDDA through melt condensation at 140° C. for 6 h under vacuum (10 mbar). Also, P(SA-RA) and P(SA-HSA) were prepared by the reaction of PSA with RA and HSA using a 30:70 weight ratio. The synthesis involved the esterification reaction of RA or HAS onto PSA to form carboxylic acid terminated oligomers, followed by anhydride polymerization.

### Characterization

The progress of the monomer and polymer synthesis was monitored by NMR. In addition, the structure of synthesized monomers and polymers was confirmed by NMR spectroscopy. In ¹H NMR of RA, the characteristic pendent peak at 3.62 ppm is observed for CH—OH. Also, the double bond protons are observed at 5.54 ppm and 5.40 ppm. When RA reacted with succinic anhydride, the characteristic pendent peak of RA at 3.62 ppm shifts to 4.90 ppm in RAS. In addition, two new peaks for succinate CH2 protons are detected at 2.67 ppm and 2.61 ppm. During the activation of RAS diacid with acetic anhydride, the peaks at 2.34 ppm and 2.22 ppm for CH3 confirm acetylation. The absence of acetylated CH3 peaks at 2.34 ppm and 2.22 ppm in the final polymer PRAS confirms the completion of polymerization. The characteristic stretching frequency at 1702 cm⁻¹ corresponds to the C═O (acid) of RA. After the reaction of RA with succinic anhydride, the formation of RAS is confirmed by the appearance of a sharp C═O(ester) band at 1732 cm⁻¹. Then, the ester-diacid was polymerized, and poly(ester-anhydride) was confirmed by the characteristic bands at 1819 cm⁻¹ and for 1760 cm⁻¹ for C═O (anhydride) of PRAS.

The molecular weight of the polyanhydrides was determined using GPC. The polyanhydrides were obtained in the molecular weight ranges from 7981 Da to 23613 Da. The lesser molecular weight was observed for PRAP and PHSAP due to the steric hindrance of phenyl moiety near to active site acid. PHOAS exhibits the least molecular weight among all the polyanhydrides due to the steric hindrance of long side chain present in the vicinity to both active site acids.

TABLE 4
Molecular weight of synthesized poly(ester-anhydride)s analyzed by GPC

| No. | | Molecular weight | |
|---|---|---|---|
| (PDI) | Poly(ester-anhydride) | (Mw) Da | Polydispesity |
| 1 | PRAS | 14654 | 1.83 |
| 2 | PRAM | 11876 | 1.87 |
| 3 | PRAP | 8391 | 1.86 |
| 4 | PHSAS | 19081 | 2.44 |
| 5 | PHSAM | 23613 | 2.69 |
| 6 | PHSAP | 11428 | 1.96 |
| 7 | PHOAS | 7981 | 2.65 |

Samples (˜2 mg) were dissolved in 2 mL CHCl₃ (HPLC grade). GPC was performed using column with a molecular-weight range of 50-100K Da. The molecular weights were determined relative to polystyrene standards.

### Stability Studies

### Storage Stability

Generally, poly(ester-anhydride)s are unstable at room temperature. A sharp decline in molecular weight has been observed at room temperature in the previous reports. The molecular weight of polyanhydrides was stable only for one month and declined to about one third after 6 months at 4° C. In addition, they were stable merely for a few days at room temperature. Due to this instability, RT poses a practical problem with storage and handling. 6 Reported block and random (SA-RA) copolymer was unstable at room temperature. ? They had blocks of SA units along the chain, which makes it vulnerable to rapid anhydride interchange. Thus, when polyanhydrides are stored at room temperature, a sharp decline in MW is noticed. However, recently reported alternating P(SA-RA) (weight ratio 30:70) copolymer exhibits stable molecular weight for 18 months. RA side chains of alternate RA-SA polymer obstruct anhydride interchange and hydrolytic degradation by steric hindrance.

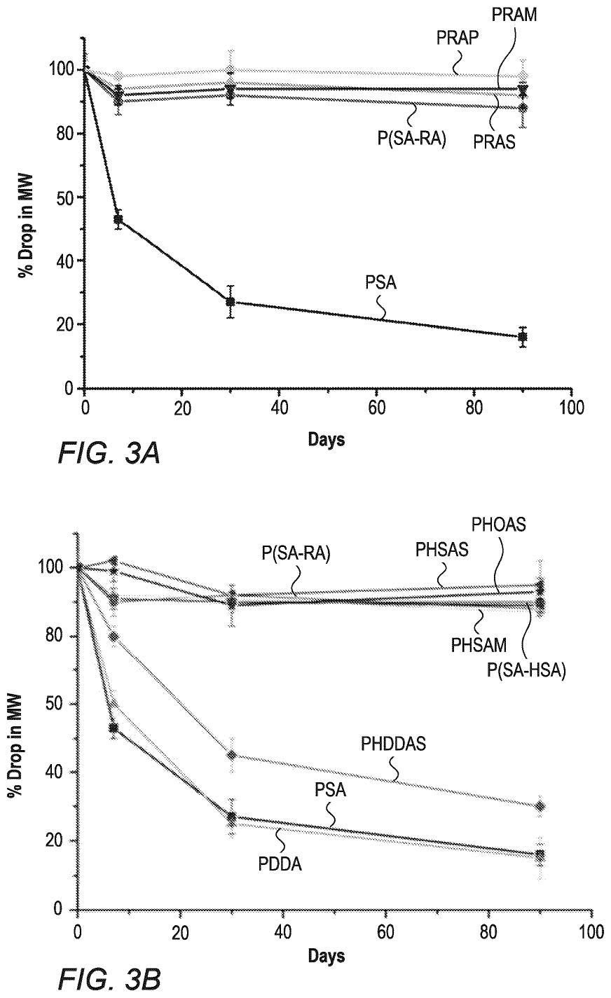

FIG. 3 presents in a diagram storage stability of polyanhydrides at room temperature for 3 months. Thus, FIG. 3A shows the relative stability of ricinoleic acid based polyanhydrides with PSA and P(SA-RA). FIG. 3B shows the relative stability of polyanhydrides with PSA and P(SA-RA) from hydroxy acid with different chain lengths. The molecular weights are an average of two independent observations (n=2). Error bars represent the percentage error calculated from an average of two observations.

In this study polyanhydride samples were packed under dry nitrogen in sealed tubes. Then, the polymer samples were stored at room temperature (˜25° C.) for three months. At each time-point (7 days, 1 month and 3 months), GPC analysis was done to determine the change in molecular weight. The results were compared with PSA and alternating P(SA-RA) (weight ratio 30:70). The molecular weight of the tested poly(ester-anhydride)s with a shorter chain length compared to P(SA-RA) was constant for three months (FIG. 3). The side chain present in the closer vicinity to the anhydride bonds offers improved stabilization hindering hydrolytic cleavage and anhydride interchange. This essential storage stability permits ease of handling and formulation of poly(ester-anhydride)s for drug delivery under common conditions. FIG. 3 shows the stability comparison of the poly(ester-anhydride)s with PSA and P(SA-RA).

### Hydrolytic Degradation Studies

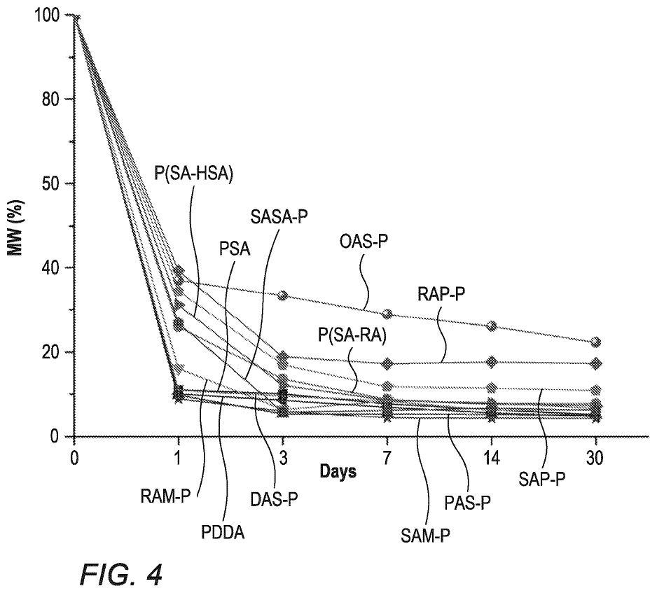

The synthesized polyanhydrides were analyzed for hydrolytic degradation and results were compared with PSA and P(SA-RA). The molecular weight (Mw) change of the polyanhydrides was measured at each time point (after 1 day, 3 days, 7 days, 14 days, and 30 days) by performing GPC analysis. The results are given in FIG. 4. FIG. 4 provides a diagram presenting hydrolytic stability results of polyanhydrides in phosphate buffer pH 7.4 at 37° C. Polymer samples were taken at the regular time point, and molecular weight was determined by GPC. An average of two independent observations (n=2) of molecular weights was used. The rate of hydrolysis of novel poly(ester-anhydrides) is slower when compared with PSA. As we reported previously, poly(ester-anhydride)s undergo hydrolytic degradation in two stages. At first the anhydride bonds of the poly(ester-anhydride) are cleaved, quickly releasing the diacid units, followed by the slow degradation of oligo-esters. After the first day, seven polyanhydrides (P(SA-RA), P(SA-HAS), PRAM, PRAP, PHSAS, PHSAP, and PHOAS) exhibit higher MW than PSA. After 3 days, five polyanhydrides (P(SA-RA), P(SA-HAS), PRAP, PHSAP and PHOAS) show higher MW than PS A. after 7, 14, and 30 days of GPC analysis, three polyanhydrides (PRAP, PHSAP, and PHOAS) still display better MW than PSA and P(SA-RA). These results reveal that the phenyl moiety present in PRAP and PHSAP reduces the hydrolysis of anhydride bonds. PHOAS demonstrates the highest MW of all the tested polymers and exhibits a moderate change from 1 day to 30 days. MW stays around 30%. This clearly shows that the presence of a side chain closer to the anhydride bond significantly decreases hydrolysis.

### Summary of the Results

The alternating architecture and hydrophobic side chains of P(SA-RA) hinder hydrolytic cleavage and anhydride interchange. We designed alternating architecture by the polymerisation of ester-diacids prepared from ricinoleic or other hydroxy acids with anhydrides such as succinic-, maleic- and phthalic anhydrides. In addition, the hydrophobic side chains are designed closer to anhydride bonds to improve hindrance to hydrolytic cleavage and anhydride interchange. The series of poly(ester-anhydrides) such as PRAS, PRAM, PRAP, PHSAS, PHSAM, PHSAP, PHOAS and PHDDAS was synthesised to investigate the effect of ester bonds, hydrophobic side chains, phenyl moieties and their distance from anhydride bonds on their stability and properties. In the first step, hydroxy acid is converted into ester-diacid by esterification reaction with anhydrides. Then, the ester-diacid is activated using acetic anhydride. Finally, the poly(ester-anhydrides) are obtained by melt condensation. PSA, PDDDA, P(SA-RA) and P(SA-HSA) were used to compare the stability and properties. The reaction progress and structure of the monomer and polymer were monitored by NMR and FT-IR. The molecular weight of the polyanhydrides was determined using GPC. The polyanhydrides were obtained in the excellent molecular weight range. Polyanhydrides were investigated for their storage stability at room temperature (˜25° C.) in a nitrogen atmosphere for 3 months using GPC, and the results were compared with PSA and P(SA-RA). The molecular weight of the tested poly(ester-anhydrides) with a shorter chain length compared to P(SA-RA) was stable for three months. Polyanhydrides were analyzed for hydrolytic degradation studies by performing GPC analysis. These results reveal that the phenyl moiety present in PRAP and PHSAP reduces the hydrolysis of anhydride bonds. PHOAS demonstrates the highest MW of all the tested polymers. The results show that the presence of hydrophobic side chains, phenyl moieties, and their distance from anhydride bonds significantly decreases the hydrolysis.

### Example 6: A Procedure for Synthesizing Poly(SA-RA) (30:70)

The polymer was synthesized in a one-pot processes using the following materials: sebacic acid, acetic anhydride and ricinoleic acid that was prepared from the hydrolysis of castor oil.

TABLE 5
Exemplary steps for the synthesis of a polymer of the invention

| | TEMP | |
|---|---|---|
| STEP | (° C.) | PROCESS DESCRIPTION |
| 1 | Room | In 500 ml round bottom flask (RBF) equipped with |
| | temp. | magnetic stirrer add 30 g of Sebacic acid and 30 g of |
| | | Ricinoleic acid |
| 1a | 175° C. | heat and mix for 5 hours |
| 1b | 175° C. | Add another 30 g of RA, close the RBF under purged |
| | | N2 and continue mixing for additional 4 hours |
| 1c | 175° C. | Add another 10 g of RA, close the RBF and continue |
| | | mixing for additional ~16 hours (overnight) under |
| | | vacuum |
| 1d | Room | Keep a sample of the obtained SA-RA oligomers |
| | temp. | mixture for MS and NMR analysis |
| 2 | Room | Add 32.6 mL of acetic anhydride (1 equivalent with |
| | temp. | respect to sebacic acid groups) to the SA-RA oligomers |
| | | mixture |
| 2a | Room | Filter the solution thought 0.2μ m filter membrane |
| | temp. | |
| 2b | 140° C. | Connect the flask to reflux condenser, warm to reflux |
| | | for 2 hours, evaporate the residual acetic anhydride or |
| | | acetic acid under reduced pressure. |
| 2c | 160° C. | Melt condensation polymerization: increate the |
| | | temperature to 160° C. and continue high vacuum for |
| | | 4 hours to affect polymerization. High vacuum |
| | | (<1 mmHg) should be used to effect polymerization. |
| | | Check temperature to remain constant. |
| 2d | Room | Weigh the flask to calculate yield. Product should be |
| | temp. | kept under N2 in a close container. |
| 2e | Room | Final product analysis: GPC, FTIR, NMR, paste |
| | temp. | viscosity (injectability) |
| | | Additional analysis (optional): residual Acetic |
| | | anhydride by LC-MS/GS-MS, Iodine number |

### Example 7: Synthesis of Poly(Ester-Anhydrides) with Controlled Molecular Weight

Abstract: Here we report the synthesis of a series of poly(ester-anhydride) composed of the following dicarboxylic acids: adipic acid, suberic acid, sebacic acid, dodecanedioic acid; and hydroxyalkanoic acids: ricinoleic acid, 12-hydroxystearic acid with control molecular weight approaching from a new method where well-defined ester monomers are first synthesized by stepwise addition of hydroxyalkanoic acids molecules into melted dicarboxylic acids. The key advantage of the synthesis of ester monomers in this modified method is the full consumption of hydroxy acid devoid of any self-condensation. The ester monomers are then activated using variable amounts of acetic anhydride followed by melt condensation polymerization. The molecular weights of the synthesized poly(ester-anhydride) are highly controllable depending upon the degree of activation of the ester monomers; hence the amount of acetic anhydride. All the synthesized poly(ester-anhydrides) are thoroughly characterized by ¹H NMR, FTIR, and GPC.
Introduction: Poly(ester-anhydrides), synthesized from dicarboxylic acids and hydroxyalkanoic acids, displays slow degradation as the hydrophobic nonlinear side chains of hydroxy acid shielded the hydrolytic cleavage of anhydride linkage. Furthermore, the degradation of these poly(ester-anhydrides) can be well controlled by the manipulation of the composition of the polymer.

Complete esterification of hydroxyalkanoic acids with the dicarboxylicacids without any self-condensation is the key criteria for the synthesis of ester monomers to avoid rapid hydrolysis and instability of the final poly(ester-anhydride), generally synthesized by one-pot melt polycondensation. Poly(ester-anhydrides) with controlled molecular weight is essential, for any specific applications where superior physicomechanical properties are required.

The objective of this study is to develop a general synthesis procedure for making different kind well-defined ester monomers and poly(ester-anhydrides) with proper control over their molecular weight for desired properties as improved biodegradable polymers. Poly(ester-anhydrides) composed of the diacids: adipic acid (AA), suberic acid (SUA), sebacic acid (SA), dodecanedioic acid (DA); and hydroxy acids: ricinoleic acid (RA), 12-hydroxystearic acid (HSA) were synthesized by melt polycondensation with different amounts of acetic anhydride.

### Materials

Adipic acid (99% pure; Fluka, Buch, Switzerland), Suberic acid (98%, Sigma-Aldrich), Sebacic acid (99% pure; Sigma-Aldrich, Rehovot, Israel), Dodecanedioic acid (99%, Sigma-Aldrich), 12-hydroxystearic acid (Tokyo Chemical Industry Co., Ltd., Japan), and acetic anhydride (Biolab, Jerusalem, Israel) were used as received. All the solvents were of analytical-grade from Biolab (Jerusalem, Israel) and were used without further purification. Ricinoleic acid (RA) was prepared from the hydrolysis of castor oil (Eur. Ph; Haifa, Israel) as described in the synthesis part.

### Spectral Analysis:

1H spectra were obtained on a Varian 300 MHz NMR spectrometer in tubes with 5 mm external diameter. CDCl₃ used as a solvent containing tetramethylsilane as shift reference.

Fourier transform infrared (FTIR) spectroscopy was performed using a Smart iTR ATR sampling accessory for Nicolet iS10 spectrometer with a diamond crystal (Thermo Scientific, Massachusetts).

### Molecular Weight Determination:

The molecular weights were determined by gel permeation chromatography (GPC) system consisting of a Waters 1515 isocratic HPLC pump with a Waters 2410 refractive index detector, a Waters 717 plus autosampler, and a Rheodyne (Cotati, CA) injection valve with a 20 μL-loop. The samples were eluted with CHCl₃ (HPLC grade) through linear Styragel HR5 column (Waters) at a flowrate of 1 mL/min. The molecular weights were determined relative to polystyrene standards (Polyscience, Warrington, PA).

### Synthesis and Characterization:

### Ricinoleic Acid

In a 1000 mL round bottom flask, 48 g of KOH was dissolved in 400 mL of ethanol by heating (65° C.). Then 200 g of castor oil was added to it and mixed them properly. The mixture was then refluxed for 2 hr at 140° C. with continuous staring. After the reflux, the solvent was evaporated by rota. Then 200 mL of double distilled water, 150 mL diisopropyl ether, and 150 mL H₃PO₄ were added and the total mixture was transferred to a separating funnel. It was then repeatedly washed with double distilled water (3-5 times, 200 mL each time) until the pH of the aqueous phase ˜4. Then the organic phase was collected through sodium phosphate and evaporated by rotavap to get pure 185 g of ricinoleic acid (yield 92.5%), confirmed by ¹H NMR. ¹H NMR (300 MHz, Chloroform-d) δ 5.56 (dt, J=10.8, 7.4 Hz, 1H), 5.47-5.30 (m, 1H), 3.62 (p, J=6.1 Hz, 1H), 2.34 (t, J=7.4 Hz, 2H), 2.21 (t, J=6.9 Hz, 2H), 2.04 (q, J=6.8 Hz, 2H), 1.63 (p, J=7.2 Hz, 2H), 1.48-1.43 (m, 2H), 1.37-1.20 (m, 16H), 0.88 (t, J=6.0 Hz, 3H); FTIR (cm⁻¹) 3008, 2924, 2854, 1708, 1457, 1410, 1244.

Ester Monomers of Dicarboxylic Acids (AA/SUA/SA/DA) and Hydroxyalkanoic Acids (RA/HSA)

The ester monomers were synthesized by heating dicarboxylic acid and hydroxyalkanoic acid at 160° C. In a round bottom flask, 30 g of dicarboxylic acid (adipic acid/suberic acid/sebacic acid/dodecanedioic acid), 30 g of hydroxyalkanoic acid (ricinoleic acid/12-hydroxystearic acid) were taken and heated to 160° C. for 3 hours under nitrogen. Then another 30 g of hydroxyalkanoic acid was injected to the round bottom flask and continued to heat for another 3 hours under nitrogen swift. Finally, another 10 g of hydroxyalkanoic acid was injected and again continued to heat for another 24 hours with mixing under vacuum (10 mbar) to yield ester monomer with 3:7 weight ratios of dicarboxylic acid and hydroxyalkanoic acid which was characterized by ¹H NMR and FTIR.

Sebacic acid-ricinoleic acid (SA-RA) and sebacic acid-12-hydroxystearic acid (SA-HSA) ester monomers of different weight ratios (1:4, 1:3, and 7:13 for SA-RA; 1:4 for SA-HSA) were also prepared by the same process and characterized by ¹H NMR and FTIR. Sebacic acid-ricinoleic acid (SA-RA) ester monomer of 3:7 weight ratio was also prepared by the single step addition of RA at the same condition (160° C., 10 mbar) and reaction time (30 hr) as a blank experiment and characterized by ¹H NMR.

### Poly(Ester-Anhydrides) of Dicarboxylic Acids (AA/SUA/SA/DA) and Hydroxyalkanoic Acids (RA/HSA):

In a typical synthesis, 10 g of each ester monomers were melted at 140° C. under nitrogen atmosphere. Then 1 or 0.7 equivalent of acetic anhydride (with respect to carboxylic acid groups) was added to the every molten ester monomers and refluxed at 140° C. for 2 hours. Any excess acetic anhydride or acetic acid was evaporated. The residue was then subjected to melt condensation polymerization at 160° C. under vacuum (˜10 mbar) for 4 hours and characterized by ¹H NMR, FTIR, and GPC.

The SA-RA ester monomer of 3:7 weight ratio was also polymerized under same procedure where different amounts (0.5, 0.35, 0.25, and 0.15 equivalent with respect to carboxylic acid) of acetic anhydride were used (refluxed at 140° C., 2 hours) to use less amount of acetic anhydride and also to make a control over the molecular weight and was characterized by FTIR and GPC.

### Results and Discussion:

Here a series of poly(ester-anhydride) copolymers is synthesized through solvent free melt polycondensation process. The synthesis involved the esterification reaction of hydroxy acids (RA/HSA) with dicarboxylic acids (AA/SA/SUA/DA) to form carboxylic acid terminated monomers, followed by anhydride polymerization to form poly(ester-anhydrides) (Scheme 1). The esterification reaction of hydroxy acids with dicarboxylic acids was particularly focused to eliminate any possibility of self condensation as well as complete consumption of hydroxy acids prior to polycondensation. Hydroxy acids consumption was quantified by sampling and recording ¹H NMR of the reaction mixture at regular intervals and monitoring the single proton of —OH group bearing carbon in RA.

Under this reaction condition (via step by step addition of RA or HAS to the dicarboxylic acids) 100% of the RA or HSA is consumed in the esterification reaction which is confirmed from the ¹H NMR spectra as the signal at 3.6 ppm (rectangle shaded area) of the single proton of —OH group bearing carbon in RA/HAS is gone astray after the final step of esterification whereas still some unreacted RA is presented in case of single step addition of RA. Furthermore self-condensation of RA or HSA in this protocol is also avoided; evidence form ¹H NMR, as there is no signal at 4.1 ppm for the proton of the selfcondensed hydroxy acids. Whereas the ester bond adjacent protons are appeared at 4.8 ppm (—COOCH—) and 2.43 ppm (—CH2COO—). These two peaks indicated the presence of ester bonds in all the monomers.

All the NMR spectra of the final ester monomers lacked a peak at 3.6 ppm which indicates no free alcohol of RA/HSA in the final product. Hence this stepwise addition protocol gives a well-defined AA-RA, SUA-RA, SA-RA, DA-RA or SA-HSA ester monomers without any residual or self-condensed RA or HSA.

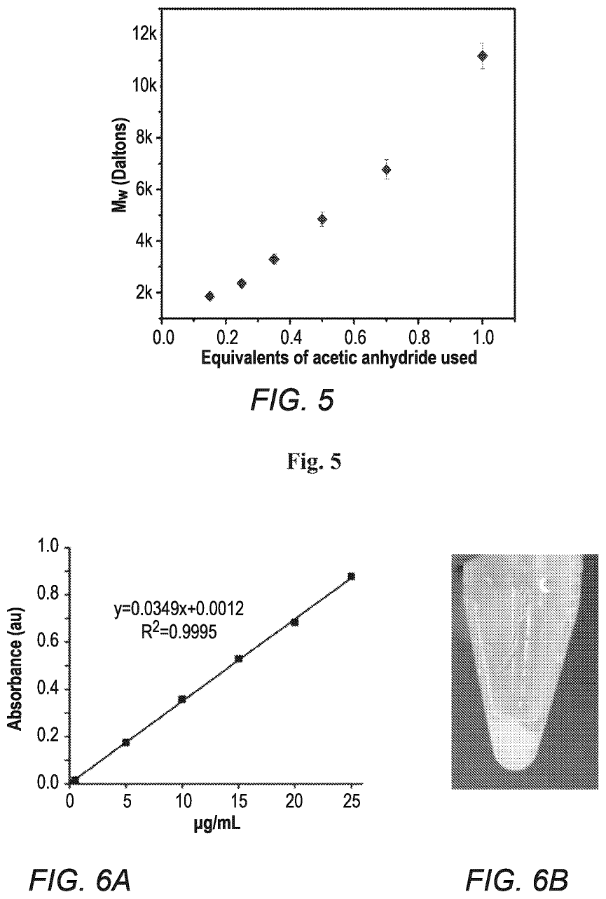

The molecular weight of the as-synthesized polymers is measured by GPC. The details of the molecular weight and disparity are given in the Table 6. From the table, it is clear that the molecular weight of polyanhydride is highly depends upon the amount of acetic anhydride is used in the activation step of monomers. Control over molecular weight of poly(ester-anhydrides) depending upon the acetic anhydride used is given in FIG. 5 where the variation of molecular weight of poly(SA-RA) with 3:7 weight ratio is plotted against the amount of acetic anhydride used during the activation of monomer. The molecular weight of the poly(SA-RA) is increases almost linearly with the increasing amount of acetic anhydride for the activation of monomer.

TABLE 6
Molecular weight of different poly(ester-anhydrides) analyzed by GPC.

| | | Equivalent of | Molecular | |
|---|---|---|---|---|
| | | acetic | weight (Mw) | |
| Sl. no | polymer | anhydride | Daltons | Disparity |
| 1 | Poly(SA-RA) | 1 | 14720 | 2.60 |
| | with 1:4 weight | 0.7 | 12745 | 2.45 |
| | ratio | | | |
| 2 | Poly(SA-RA) | 1 | 13663 | 2.63 |
| | with 1:3 weight | 0.7 | 7895 | 2.04 |
| | ratio | | | |
| 3 | Poly(SA-RA) | 1 | 9157 | 2.45 |
| | with 7:13 weight | 0.7 | 5374 | 1.91 |
| | ratio | | | |
| 4 | Poly(SA-RA) | 1 | 11175 | 2.63 |
| | with 3:7 weight | 0.7 | 6776 | 2.05 |
| | ratio | 0.5 | 4841 | 1.72 |
| | | 0.35 | 3296 | 1.51 |
| | Poly(SA-RA) | 0.25 | 2357 | 1.35 |
| | with 3:7 weight | 0.15 | 1856 | 1.24 |
| | ratio | | | |
| 5 | Poly(SA-HSA) | 1 | 13188 | 2.17 |
| | with 1:4 weight | 0.7 | 10980 | 2.09 |
| | ratio | | | |
| 6 | Poly(SA-HSA) | 1 | 9579 | 2.09 |
| | with 3:7 weight | 0.7 | 6526 | 1.82 |
| | ratio | | | |
| 7 | Poly(AA-RA) | 1 | 6351 | 2.24 |
| | with 3:7 weight | 0.7 | 3473 | 1.62 |
| | ratio | | | |
| 8 | Poly(SUA-RA) | 1 | 9035 | 2.21 |
| | with 3:7 weight | 0.7 | 6082 | 1.94 |
| | ratio | | | |
| 9 | Poly(DA-RA) | 1 | 9095 | 2.15 |
| | with 3:7 weight | 0.7 | 7334 | 1.99 |
| | ratio | | | |

Furthermore the FTIR study of poly(SA-RA) ester-anhydride with 3:7 weight ratio is also supports that the control over molecular weight of poly(ester-anhydride) depends upon the used acetic anhydride during the activation of monomer.

Conclusion: An effective route for the synthesis of aliphatic poly(ester-anhydrides) made from a series of dicarboxylic acids and saturated or unsaturated hydroxyalkanoic acids with control molecular weight is reported. Special attention was given in to the esterification reaction between the dicarboxylic acids and hydroxyalkanoic acids for the synthesis of ester monomers with full consumption and avoiding the possibility of any self-condensation of hydroxyalkanoic acids. The molecular weights of the synthesized polymers in this reported protocol are highly controllable depending upon the degree of activation of the ester monomers. This route forwarded an idea for producing aliphatic poly(ester-anhydrides) with control molecular weight for possible use in the preparation of degradable disposable medical supplies.

### Example 8: Release of Escitalopram (Cipralex) from PSA:RA Gels

Poly (sebacic-co-ricinoleic) acid (P(SA:RA)) is a pasty polymer with biodegradable properties. The polymer has hydrophobic backbone with hydrolytically labile anhydride that hydrolyze to dicarboxylic acids and hydroxy acid monomers when placed in aqueous medium. The incorporation of the varying ratios of the ricinoleic in the PSA:RA makes it a pasty polymer with desired injectability using a thin gauze syringe. Because of being a pasty polymer, various drugs, ranging from extreme hydrophobic to hydrophilic properties can be easily incorporated into the polymer matrix by simple grinding. This report describes the preparation of formulations for extended release of the high concentration of Escitalopram from the PSA:RA 3:7.

Materials: a) Escitalopram, Batch No. HWP180212, was in the lab; b) PSA:RA 3:7 (Mw 11675, PDI 2.63) was synthesized in the lab.

### Method

Formulation preparation: The formulations of P(SA:RA) 3:7 with 10%, 20% and 30% w/w of Escitalopram were prepared by direct mixing of the polymer with the drug at room temperature. The final pasty formulation was loaded in 1 mL syringe. The obtained formulations were injectable semisolid pastes at room temperature.
In vitro drug release: In vitro drug release was conducted by injecting the pasty formulations sample in a 4 mL phosphate buffer solution (10 mM, pH 7.2) at 37° C. with constant shaking (40 RPM). The samples were analyzed in duplicates. The paste hardened to a soft solid shortly after addition to the buffer. The release medium was replaced periodically with fresh buffer solution, and Escitalopram concentration in the solution was determine using drug absorbance at 238 nm. FIG. 6, shows the representative graph of plotting standard curve for Escitalopram in buffer (FIG. 6).
Result: The presence of Escitalopram in the PSA:RA did not affect its injectable properties. Further, the formulations became hard gels instantly after addition of the buffer medium. The appearance of the PSA:RA Escitalopram formulation is white due to the white appearance of the Escitalopram.

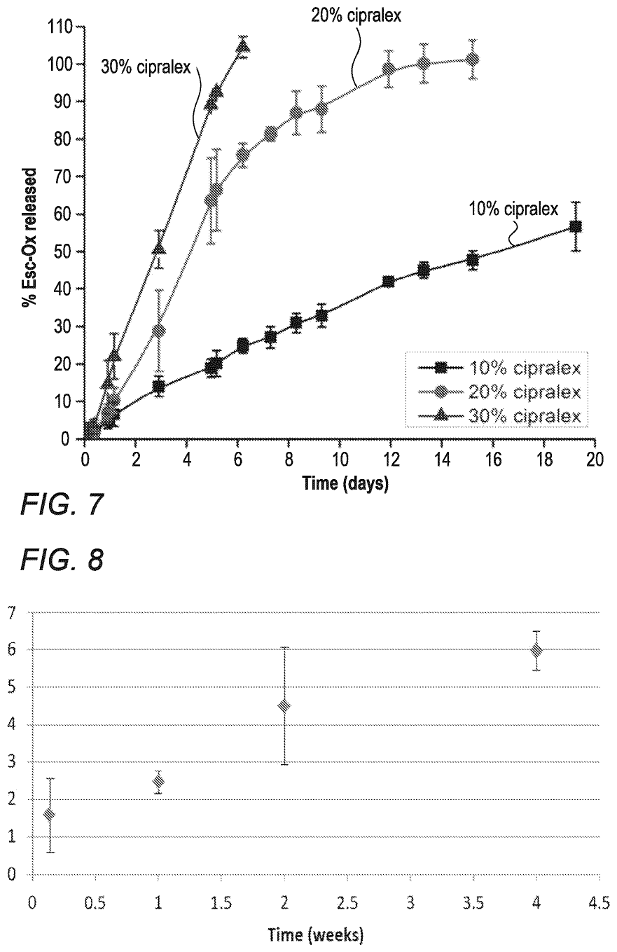

Incorporation of 10%, 20% and 30% (w/w) of Escitalopram in the PSA:RA 3:7 by mixing at room temperature had no initial burst effect, however, the release of the Escitalopram is dependent on its amount in the PSA:RA gel. The release of the drug is faster with 30% drug formulation and gradually decreases with the decrease with the amount of the drug in the PSA:RA gel. Apparently, an extended release is observed in 10% (w/w) Escitalopram formulation for more than 19 days where up to 60% of the drug is released (FIG. 7). Summary of the Results: The presence of high amount of Escitalopram in PSA:RA does not influence its gelling properties. Further, there is no burst release observed in any of the formulations immediately after addition of the release medium at 37 C. 10% drug formulation show slow release because of the high amount of hydrophobicity due to the polymer, which does not allow the surrounding solvent to penetrate deep into the gel. Additionally, in all the formulations tested, the PSA:RA gel remained intact despite the amount of cipralex content, and did not spread out or disintegrated at any time point of release. This is an indication of the surface degradation properties of the PSA:RA which readily controls the release of the hydrophilic drugs in water. Meaning, if the polymer content is high, a slow release is observed.

Insulin delivery: This example illustrates the ability of the invented polymer to allow constant release of protein-based drug: The formulation consist of 0.03% Insulin in PSA-RA. The preparation was done by lyophilization of insulin and mannitol to reach mixture of 10% of insulin. This mixture was added, geometrically, with additional amount of Mannitol, and then mixed geometrically with appropriate amount of PSA-RA to get the 0.03% of total insulin in formulation.

FIG. 8 In vitro release study was preformed of PSA-RA duplicate formulation. Each monolete was 300 mg of formulation and contained 90 ug of insulin. Release medium was phosphate buffer pH 7.4. The results demonstrated that 6% of the total amount of insulin was released along 4 weeks.

The polymers of this invention, particularly the poly(RA-SA)70:30 was tested in vivo in rate for drug release, toxicity elimination and was found to release the drug for 30 days while being mostly eliminated from the site of injection within 8 weeks. No side effects in rat general health during the study, all gained weight similar to the control rats, no swelling or irritation at the site of polymer administration and necropsy indicated normal internal tissues with minimal adverse effect in the injection site.

### Example 9: Molecular Weight Determination of P(RA:SA) 75:25 Pasty Polyanhydrides

In this study, a combination of sebasic acid and ricinoleic acid (25:75) were used to synthesis two sets of polymers. The first set of P(SA-RA) includes different polymerization times such as 2, 4, 6 and 8 hrs. by activation using 0.9 equiv. of acetic anhydride, and the second set of P(SA-RA) includes activation using different ratios of acetic anhydride of 0.7, 0.8, 0.9 and 1.0 equiv. at 3 hrs. of polymerization time. Three samples were synthesized from each polymer to verify the reproducibility of the data. The samples were checked for their molecular weight and viscosity.

### Materials and Methods

Sebacic acid (SA, 99% pure; Aldrich, Milwaukee, WI), and acetic anhydride (Merck, Darmstadt, Germany) were used as received. Ricinoleic acid (RA) was prepared from the hydrolysis of castor oil, P(SA-RA) 25:75 samples were synthesized as described above.

### Gel Permeation Chromatography (GPC)

The molecular weights were determined by gel permeation chromatography (GPC) system. samples were analyzed using a Refractive Index detector (RI detector 410, 40 C, USA) with Waters 717 plus Autosampler (USA) and Waters Pump. The system was equipped with the Styragel HR4E column (5 μm, mixed bed, 7.8 mm×300 mm, MW 50-100K, THF). The mobile phase was chloroform, flow 1 mL min⁻¹, injection volume 20 pt and 15 min run time per sample.

### Calibration Curve

In order to calculate the molecular weight of the polyanhydride samples, the calibration curve was obtained using standard polystyrene samples. Polystyrene standards (standards with the molecular weight of Mp: 1820, Mp: 3470, Mp: 5440, Mp: 28000, Mp: 54000 and Mp: 93800) were dissolved in in chloroform directly into the GPC vials. The retention time of the polystyrene standards was used to make the calibration curve, which was used to estimate the molecular weight of the unknown polyanhydrides samples.

### Polymer Samples

10 mg of each P(SA-RA) 25:75 sample were accurately weighed in 4 mL vials and then dissolved in 2 mL of chloroform and vortexed. After samples were completely dissolved in chloroform the solutions were filtered over cotton plug directly into GPC vials. Each sample was prepared and analyzed separately because the samples are unstable when stored at room temperatures. The data that have been obtained are weight average molecular weight (Mw), number average molecular weight (Mn) and peak molecular weight (Mp).

### Viscosity by Ubbelhode-Type Viscometer

Viscometry of the polymers in chloroform was measured in Cannon-Ubbelohde 50 μm dilution viscometer. Afflux times were measured at three concentrations at 25° C. 40 mg of P(SA-RA) polymers with different polymerization times such as 2, 4, 6 and 8 hrs. by activation using 0.9 equiv. of acetic anhydride, and 40 mg of P(SA-RA) includes activation using different ratios of acetic anhydride such as 0.7, 0.8, 0.9 and 1.0 equiv. at 3 hrs. of polymerization time were approximately weighted and dissolved in 3 mL chloroform. The samples were diluted directly in the viscometer twice and the viscosity was measured.

### Viscosity by Rheometer

P(SA-RA) 25:75 samples from two sets of experiments were checked for their viscosity. The first experiment includes different polymerization times such as 2, 4, 6 and 8 hrs by activation using 0.9 equiv. of acetic anhydride, and the second experiment includes activation using different ratios of acetic anhydride such as 0.7, 0.8, 0.9 and 1.0 equiv. at 3 hrs of polymerization time.

Polymer viscosity and rheological behavior were performed using an Anton Paar plate on plate Physica MCR101 rheometer with a measuring plate PP25, diameter 25 mm. Samples were measured at shear rates from 0.01 to 100 second⁻¹ at 25° C. The zero-gap was set up at 0.5 and measurements were conducted in triplicates at 25° C.

### Amplitude Sweep Test

An oscillatory test was applied to determine the rheological behaviour of the sets of P(SA-RA) 25:75 samples that were prepared on our lab using an Anton Paar plate on plate Physica MCR101 rheometer with a measuring plate PP25, diameter 0.1 mm and it was set at 25 C °. an amplitude sweep test was performed from 0.1 to 100 amplitude gamma at a constant angular frequency of 10 rad[s]⁻¹. The measurements were conducted in triplicate.

### Results and Discussion

Two sets of p(SA-RA) 25:75 polymers. The first includes different polymerization times such as 2, 4, 6 and 8 hrs. by activation using 0.9 equiv. of acetic anhydride, and the second set of P(SA-RA) includes activation using different ratios of acetic anhydride such as 0.7, 0.8, 0.9 and 1.0 equiv. at 3 hrs. of polymerization time. Three samples were synthesized from each polymer in order to verify the reproducibility of the data. The samples were checked for their molecular weight and viscosity and a relation between the molecular weight and viscosity was noticed.

### Results of Gel Permeation Chromatography (GPC)

The samples were analyzed using a Refractive Index detector (RI detector 410, 40 C, USA) with Waters 717 plus Autosampler (USA) and Waters Pump. The system was equipped with the Styragel HR4E column (5 μm, mixed bed, 7.8 mm×300 mm, MW 50-100K, THF). The mobile phase was chloroform, flow 1 mL min⁻¹, injection volume 20 μL and 15 min run time per sample.

The molecular weights of the polymers with different ratios of acetic anhydride such as 0.7, 0.8, 0.9 and 1.0 equiv. at 3 hrs. of polymerization time increase as the acetic anhydride's ratio increases. Moreover, the molecular weights of P(SA-RA) 25:75 with different polymerization times 2,4,6 and 8 hrs. increase as the polymerization time increases with no significant difference between 6 hrs. and 8 hr.s polymerization time.

Viscometry is one of the simplest and most rapid methods for measuring the molecular weight of polymers. This method is based on the well-known Mark-Houwink equation:

[n]=kM^{α}

The values of k and α in the Mark-Houwink equation have been determined for the P(SA-RA) 25:75 polymers. The weight average molecular weight (Mw) of P(SA-RA) 25:75 polymers samples was determined by GPC and the intrinsic viscosity [η] of the samples was measured with an Ubbelohde viscometer. The plot of log Mw versus log [η] for all the P(SA-RA) 25:75 samples gave straight line when the slope refers to α value and the intercept refers to k value. Table 7 shows the intrinsic viscosity values that obtained from ubbelohde viscometer and the Mw values obtained from GPC. The Mark Houwink equation parameters for P(SA-RA) polymers 25:75 were determined by Mw values that obtained from GPC and viscosity measurements that obtained from Ubbelohde type viscometer when α=0.6692 and k=0.000275 [dL/gr]. The polydispersity for most samples was between 1.4 and 2.1.

TABLE 7
Mw values obtained fron GPC and intrinsic viscosity values from
Ubbelohde viscometer for P(SA-RA) 25:75 polymers with different
polymerization times: 2, 4, 6 and 8 hrs. by activation using 0.9 equiv. of
acetic anhydride and P(SA-RA) 25:75 polymers using different ratios of
acetic anhydride such as 0.7, 0.8, 0.9 and 1.0 equiv. at 3 hrs. of
polymerization time.

| | Intrinsic visc. (η) | Mw (GPC) |
|---|---|---|
| | [dL/gr] | [gr/mol] |
| 0.7 equiv.1 | 0.1266 | 11121 |
| 0.7 equiv.2 | 0.134 | 11885 |
| 0.7 equiv.3 | 0.1325 | 11457 |
| 0.8 equiv.1 | 0.128 | 13222 |
| 0.8 equiv.2 | 0.1709 | 13063 |
| 0.8 equiv.3 | 0.1605 | 13135 |
| 0.9 equiv.1 | 0.1959 | 15077 |
| 0.9 equiv.2 | 0.2027 | 17731 |
| 0.9 equiv.3 | 0.1915 | 15757 |
| 1.0 equiv.1 | 0.2121 | 16245 |
| 1.0 equiv.2 | 0.1624 | 18757 |
| 1.0 equiv.3 | 0.1920 | 17386 |
| 2 HR-1 | 0.1602 | 13878 |
| 2 HR-2 | 0.1615 | 13987 |
| 2 HR-3 | 0.1688 | 13842 |
| 4 HR-1 | 0.1973 | 14261 |
| 4 HR-2 | 0.1821 | 14810 |
| 4 HR-3 | 0.1833 | 15060 |
| 6 HR-1 | 0.1453 | 16700 |
| 6 HR-2 | 0.1996 | 17059 |
| 6 HR-3 | 0.1577 | 18256 |
| 8 HR-1 | 0.2179 | 16870 |
| 8 HR-2 | 0.2342 | 16368 |
| 8 HR-3 | 0.2539 | 16543 |

### Results of Viscosity Measurements by Rheometer

The polymer viscosity was tested in a plate on plate rheometer. Testing was done in triplicate. viscosity was measured under increasing shear rates. As the molecular weight increased within a polymer series, the viscosity increased dramatically. Data are expressed as viscosity versus shear rate. The viscosity values increase as the polymerization time increases. In addition, the viscosity values increase as the activation ratio increase due to the increase in the molecular weight of the polymer. The rheological behavior of the polymer is expressed as viscosity/shear rate. Viscosity results of the polymer analyzed show that the p(SA:RA) 25:75 follows non-Newtonian behavior. As a result, the polymer behaves like a pseudo-plastic shear-thinning material; as the shear rate increases, the pasty polymer reduces its viscosity, becoming softer.

### Results of Amplitude Sweep Test

The data obtained through oscillatory measurements are the contributions to the internal structure of the sample from the elastic and viscous portions of flow, G′ and G″″ (Pa), respectively. The storage modulus G′ (in Pa) represents the elastic portion of the viscoelastic behavior, which describes the solid-state behavior of the sample. The loss modulus G″ (in Pa) characterizes the viscous portion of the viscoelastic behavior, which can be seen as the liquid-state behavior of the sample.in other words, G″ is the viscous contribution to flow. Storage modulus (G′) represents is the energy stored per deformation cycle during an oscillatory test. It is related to the stiffness of the network. Loss modulus (G″) characterizes the deformation energy lost or dissipated through internal friction when flowing. Viscoelastic solids with G′>G″ have a higher storage modulus than loss modulus. This is due to links inside the material like chemical bonds or physical-chemical interactions. On the other hand, viscoelastic liquids with G″>G′ have a higher loss modulus than storage modulus. The reason for this is that, in most of these materials, there are no such strong bonds between the individual molecules.

When analyzing the results from amplitude sweeps, the values of G″, G″ increased as the polymerization time increases and as the activation ratio increases due to the increase in the molecular weight of the polymer. The P(SA-RA) 25:75 polymers showed a predominantly viscous behavior (G″>G′) over the whole range of amplitudes tested, which corresponds closely to liquid. The same results obtained from P(SA-RA) 25:75 polymers with different ratios of acetic anhydride such as 0.7, 0.8, 0.9 and 1.0 equiv. at 3 hrs. and P(SA-RA) 25:75 with different polymerization times 2,4,6 and 8 hrs.

### CONCLUSIONS

Two sets of P(SA:RA) 25:75 polymers were synthesized: (1) P(SA-RA) polymers with different polymerization times, 2, 4, 6 and 8 hrs. by activation using 0.9 equiv. of acetic anhydride (2) P(SA-RA) with activation using different ratios of acetic anhydride such as 0.7, 0.8, 0.9 and 1.0 equiv. at 3 hrs. of polymerization time. The synthesis procedure modifies the physicochemical characteristics of the pasty polymer. polymer viscosity was shown to increase with increasing molecular weight. Moreover, the polymers displayed non-Newtonian pseudoplastic flow behavior. The values of k and a of the Mark-Houwink equation were determined and can be used to calculate the molecular weight of P(SA-RA) 25:75 polymers. P(SA-RA) 25:75 polymers have viscous behavior where G, G″ and viscosity are higher in polymers with higher molecular weight.

### Example 10: Branched and Crosslinked Polyanhydrides

Polymers described in the examples above are linear chains. Branching and crosslinking of these polymers is achieved by adding a tricarboxylic acid molecule or a polycarboxylic acid molecule to the dimer-trimer oligomer mixture, prior to the anhydride acetylation with acetic anhydride. Examples of polycaboxylic acid molecules include citric acid, aconitic acid, isocitric acid, Propane-1,2,3-tricarboxylic acid, agaric acid, benzene-tricarboxylic acid, polyacrylic acid, and 1,2,3,4,5,6-Cyclohexanehexacarboxylic acid. The amount added can be from 0.1 to about 5% to form a branched polymer with higher viscosity compared to the linear polymer or form a crosslinked polymer when using 5% or more polyacid molecules. The physical and mechanical properties of the polymers are affected by the degree of branching or crosslinking. In a typical experiment, 1 gram of isocitric acid is mixed with 99 grams of dicarboxylic acid mixed oligomers prepared from the reaction of sebacic acid and ricinoleic acid at a 30:70 w/w ratio. The mixture is reacted with a one mole equivalent per carboxylic acids of acetic anhydride and polymerized to form a branched polymer. Alternatively, isocitric acid is added to the sebacic acid and ricinoleic acid to form trimers and dimers oligomers that have tricarboxylic acids units that serve as branching molecules.

1-51. (canceled)

52. A process for producing a narrow-polydisperse polyanhydride, the process comprising melt polycondensation of a dicarboxylic acid and a hydroxy-alkanoic acid in the presence of an amount of acetic anhydride not exceeding a mole equivalent thereof per each free carboxylic acid group and in absence of a solvent, wherein the process excludes formation of poly(dicarboxylic acid).

53. The process according to claim 52, the process comprising:
reacting a dicarboxylic acid (DA) or a cyclic anhydride with a hydroxy-alkanoic acid (HA) under conditions permitting esterification of the dicarboxylic acid to obtain a mono ester of the dicarboxylic acid and/or a di-ester thereof or a mixture thereof; and
transforming the esterified mono or di-ester or the mixture of the dicarboxylic acid into the narrow-polydisperse polyanhydride.

54. The process according to claim 52, wherein the dicarboxylic acid (DA) is a dicarboxylic acid of a C₆-C₁₅alkylene or alkenylene.

55. The process according to claim 52, wherein the dicarboxylic acid is selected from succinic acid, adipic acid, maleic acid, suberic acid, sebacic acid (SA), decandioic acid, azelaic acid, undecanedioic acid, dodecanedioic acid, brassylic acid and phthalic acid.

56. The process according to claim 52, wherein the hydroxyl-alkanoic acid is selected from ricinoleic acid (RA), hydroxystearic acid (HSA), hydroxyoctanoic acid, hydroxydecanoic acids, hydroxydodecanoic acid, lactic acid, glycolic acid, and hyroxycaproic acid.

57. The process according to claim 52, wherein the polyanhydride is formed of a dicarboxylic acid selected from succinic acid, adipic acid, maleic acid, suberic acid, sebacic acid (SA), decandioic acid, azelaic acid, undecanedioic acid, dodecanedioic acid, brassylic acid and phthalic acid and a hydroxy-alkanoic acid selected from ricinoleic acid (RA), hydroxystearic acid (HSA), hydroxyoctanoic acid, hydroxydecanoic acids, hydroxydodecanoic acid, lactic acid, glycolic acid, and hyroxycaproic acid.

58. The process according to claim 52, wherein the dicarboxylic acid is RA and the hydroxyl-alkanoic acid is SA.

59. The process according to claim 53, wherein the transforming of the esterified mono or di-ester or the mixture of the dicarboxylic acid into the narrow-polydisperse polyanhydride is achieved by polymerization.

60. The process according to claim 52, wherein the process comprises:
reacting the dicarboxylic acid (DA) or the cyclic anhydride with the hydroxyl-alkanoic acid (HA) at a temperature between 80 and 200° C. to obtain a mixture of a mono ester (DA-HA) and a diester (HA-DA-HA) of the dicarboxylic acid; and
reacting the mixture with acetic anhydride permitting polymerization of the mono ester and diester into the polyanhydride.

61. The process according to claim 52, the process comprises:
reacting the dicarboxylic acid (DA) or the cyclic anhydride with the hydroxyl-alkanoic acid (HA) at a temperature between 80 and 200° C. to obtain a mixture of a mono ester (DA-HA) and a diester (HA-DA-HA) of the dicarboxylic acid; and
reacting the mixture with acetic anhydride to acetylate the mixture of the monoester and diester; and
thermally treating the acetylated mixture under conditions permitting polymerization into the polyanhydride.

62. The process according to claim 52, the process comprises:
reacting the dicarboxylic acid (DA) or the cyclic anhydride with the hydroxyl-alkanoic acid (HA) in the presence of acetic anhydride at a temperature between 80 and 200° C. to obtain a mixture of a mono ester (DA-HA) and a diester (HA-DA-HA) of the dicarboxylic acid; and
thermally treating the acetylated mixture in vaccuo at a temperature between 100 and 200° C., permitting polymerization to afford the polyanhydride.

63. The process according to claim 52, the process comprising:
reacting a dicarboxylic acid (DA) or a cyclic anhydride with a hydroxy-alkanoic acid (HA) under conditions permitting esterification of the dicarboxylic acid to obtain a mono ester of the dicarboxylic acid and/or a di-ester thereof or a mixture thereof;
reacting the mono ester of the dicarboxylic acid and/or a di-ester thereof or a mixture thereof with at least one polycarboxylic acid; and
transforming the esterified mono or di-ester or the mixture of the dicarboxylic acid into the narrow-polydisperse polyanhydride.

64. The process according to claim 63, wherein the at least one polycarboxylic acid is selected from citric acid, aconitic acid, isocitric acid, propane-1,2,3-tricarboxylic acid, agaric acid, benzene-tricarboxylic acid, polyacrylic acid, and 1,2,3,4,5,6-Cyclohexanehexacarboxylic acid.

65. A polyanhydride prepared by melt condensation of a dicarboxylic acid and a hydroxyl-alkanoic acid with a mole equivalent or less of acetic anhydride per carboxylic acid group, the polyanhydride being a narrow-polydispersed polymer.

66. The polyanhydride according to claim 63, produced by a process for producing a narrow-polydisperse polyanhydride, the process comprising melt polycondensation of a dicarboxylic acid and a hydroxy-alkanoic acid in the presence of an amount of acetic anhydride not exceeding a mole equivalent thereof per each free carboxylic acid group and in absence of a solvent, wherein the process excludes formation of poly(dicarboxylic acid).

67. The polyanhydride according to claim 64, prepared by a process comprising:
reacting a dicarboxylic acid or a cyclic anhydride with an hydroxy-alkanoic acid under conditions permitting esterification of the dicarboxylic acid to obtain a mono ester of the dicarboxylic acid or a di-ester thereof; and
transforming the esterified dicarboxylic acid into the narrow-dispersed polyanhydride.

68. The polyanhydride according to claim 64, wherein the dicarboxylic acid (DA) is SA and the hydroxyl-alkanoic acid (HA) is RA.

69. The polyanhydride according to claim 63, for use as a carrier of at least one agent.

70. The polyanhydride according to claim 69, wherein the at least one agent is an active agent, a drug, a medicament, an additive, a preservative, a coloring agent, a cosmetic agent, an herbicide, an insecticide, or a fertilizer.

71. A pharmaceutical composition comprising at least one agent and a carrier in a form of a polyanhydride according to claim 63.

72. A polyanhydride of the form —(SA-RA)n-, wherein SA is sebacic acid and RA is ricinoleic acid, and wherein n is an integer between 10 and 100, prepared by melt condensation of SA and RA with a mole equivalent or less of acetic anhydride per carboxylic acid group, the polyanhydride being a narrow-polydispersed polymer.

73. A process for producing a narrow-polydisperse polyanhydride of the form -(SA-RA)n-, wherein SA is sebacic acid and RA is ricinoleic acid, and wherein n is an integer between 10 and 100, the process comprising melt polycondensation of RA and SA in presence of an amount of acetic anhydride not exceeding a mole equivalent thereof per each free carboxylic acid group and in absence of a solvent, wherein the process excludes formation of poly(sebacic acid).
